(19) United States
(12) Patent Application Publication
(10) Pub. No.: US 2025/0011876 A1
(43) Pub. Date: Jan. 9, 2025

(54) EXTRACELLULAR RNA SIGNATURES OF MUTANT KRAS(G12C) CANCERS

(71) Applicant: The Regents of the University of California, Oakland, CA (US)

(72) Inventors: Daniel KIM, Santa Cruz, CA (US); Roman REGGIARDO, Santa Cruz, CA (US); Reem KHOJAH, Santa Cruz, CA (US)

(73) Assignee: The Regents of the University of California, Oakland, CA (US)

(21) Appl. No.: 18/706,482

(22) PCT Filed: Oct. 31, 2022
(86) PCT No.: PCT/US2022/048442

§ 371 Date: May 1, 2024

Related U.S. Application Data
(60) Provisional application No. 63/399,329, filed on Aug. 19, 2022.
(60) Provisional application No. 63/274,741, filed on Nov. 2, 2021.

(51) Int. Cl. C12Q 1/6886; A61K 31/517; A61K 31/519

(52) U.S. Cl. CPC C12Q 1/6886; A61K 31/517; A61K 31/519; C12Q 2600/156

(57) ABSTRACT

Provided herein are methods for detecting a RAS pathway mutations in a subject. The methods include obtaining a biological sample from the subject, isolating nucleic acids from the biological sample, and analyzing the expression level of extracellular RNAs in the nucleic acids, wherein a differential expression level of the extracellular RNAs compared to a control sample indicates that the subject has a RAS pathway mutation.

### CROSS REFERENCE TO RELATED APPLICATIONS

This application claims priority to U.S. Provisional Application No. 63/274,741, filed Nov. 2, 2022 and U.S. Provisional Application No. 63/399,329, filed Aug. 19, 2022, which are incorporated by reference herein in their entireties.

### BACKGROUND

The RAS family of proteins—HRAS, NRAS, and KRAS—are highly conserved in eukaryotes and enable signal transduction that mediates, in addition to a plethora of other functions, cellular proliferation. This central role in enabling the expansion of cellular populations lends itself to many cancer patients harboring a mutant allele in a RAS pathway gene.

### BRIEF SUMMARY

Provided herein are methods for detecting a RAS pathway mutations in a subject. The methods include obtaining a biological sample from the subject, isolating nucleic acids from the biological sample, and analyzing the expression level of extracellular RNAs in the nucleic acids, wherein a differential expression level of the extracellular RNAs compared to a control sample indicates that the subject has a RAS pathway mutation.

### BRIEF DESCRIPTION OF THE DRAWINGS

FIGS. 1A-C show KRAS G12C inhibition decreases cell viability and increases KRAS protein abundance. FIG. 1A is a schematic flowchart of extracellular RNA extraction and sequencing from KRASG12C mutant H358 lung cancer cell line treated with clinical grade KRASG12C inhibitor AMG 510. Conditioned media of AMG 510 (0.1 μM) treated H358 were used for exosome isolation by affinity-based column and size-based filtration for downstream exRNA sequencing pipeline. FIG. 1B is a graph showing cell viability of two- and three-dimensional H358 culture analyzed after 72 hours of AMG510 treatment. Opaque points are the means calculated for each treatment concentration. FIG. 1C are images of KRAS and phosphorylated ERK (pERK) monitored with immunoblotting after 72-hour treatment of AMG 510 (0.1 μM) from indicated 2D and 3D H358 cell models. Beta-actin was used as a loading control.

FIGS. 2A-D show transcriptional landscape of KRAS G12C inhibition. FIG. 2A is a graph showing distribution of number of transcripts detected above threshold of 5 normalized counts in both exoTIC and rnaEASY platforms. Comparison of means performed with Wilcoxon-test show insignificant differences between AMG and DMSO treatment. FIG. 2B is a graph showing stacked bar plot displaying the fraction of detected transcripts annotated as protein coding, retained intron, lncRNA, or other GENCODE biotypes. FIG. 2C is a graph showing a scatter plot comparing log 2-scale fold-changes between AMG and DMSO treatment using the exoTIC (x-axis) and rnaEASY (y-axis) platforms. Colors represent GENCODE biotypes lncRNA, protein-coding, or membership in the G12C-induced gene set from Xue et al 9. FIG. 2D is a graph showing distribution of log-scale fold-changes of genes included in Hallmark gene sets enriched in both exoTIC and rnaEASY platforms, as well as the G12C-induced gene set mentioned above.

FIGS. 3A-D show KRAS G12C inhibition leads to enrichment of lncRNAs, mtRNAs and TE RNAs. FIG. 3A is a graph showing distribution of log 2-scale fold-changes of genes from GENCODE biotypes and chromosome M. FIG. 1B is a graph showing plot of first two principal components from PCA analysis of TE-aware quantification of exoTIC and rnaEASY samples. FIG. 1C is a graph showing fraction of normalized counts assigned to transcripts belonging to displayed biotypes and TE families. FIG. 1D is a graph showing scatter plot comparing log 2-scale fold-changes between TE RNA in AMG and DMSO treatment using the exoTIC (x-axis) and rnaEASY (y-axis) platforms.

FIGS. 4A-E show exRNA signatures of KRAS G12C discriminate TCGA samples from healthy tissue. FIG. 4A is a graph showing bar plot of −log 10 transformed adjusted p-value produced for each Hallmark gene set in Gene Set Enrichment Analysis across rnaEASY, exoTIC, and TCGA LUAD data sets. FIG. 4B is a graph showing upset plot quantifying overlap of upregulated genes (log 2 fold-change>=1) in rnaEASY, exoTIC, and TCGA LUAD differential expression. The labelled consensus set is used in the following panels. FIG. 4C is a heatmap with hierarchical clustering of scaled and centered count values for the 20 genes contained in the consensus overlapping set observed in B. FIG. 4D is a graph showing distribution of average expression of the consensus overlapping gene set in TCGA LUAD samples. Comparison of means with T-test demonstrates significant difference between the G12C tumor samples and the WT matched normal samples. FIG. 4E is a graph showing Kaplan-Meier survival curve using on overall survival of TCGA LUAD samples in the top-third of consensus overlapping gene set expression and the bottom-third. The Kaplan-Meier estimate produced a significant p-value.

FIG. 5A shows H358 Spheroid area estimated for each dose of AMG 510 inhibitor. FIG. 5B shows KRAS and phosphorylated ERK (PERK) monitored with immunoblotting after 72-hour treatment of AMG 510 (0.1 μM) from indicated 2D and 3D H358 cell models. Beta-actin was used as a loading control. FIG. 5C is a graph showing the top two components (% variance explained) derived from PCA of ExoTIC and exoRNeasy 2D AMG 510 and DMSO treated conditions.

FIG. 6A. is a graph showing the overall differential expression volcano plot for ExoTIC 2D EV isolation with and without AMG 510 treatment. FIG. 6B is a graph showing significantly enriched hallmark gene sets in ExoTIC 2D. FIG. 6C is a graph showing overall differential expression volcano plot for exoRNeasy 2D EV isolation with and without AMG 510 treatment. FIG. 6D is a graph showing significantly enriched hallmark gene sets in exoRNeasy 2D. FIG. 6E is a bar graph showing upset plot of unique and overlapping significantly upregulated genes (>=1 log 2FoldChange) in exoRNeasy and ExoTIC 2D conditions. FIG. 6F is a bar graph showing upset plot of unique and overlapping significantly downregulated lncRNA genes (<=−1 log 2FoldChange) in exoRNeasy and ExoTIC 2D conditions.

FIG. 7A is a graph showing scatter plot comparing significant differential expression of genes across exoRNeasy 2D and 3D conditions. FIG. 7B is a graph showing upset plot of unique and overlapping significantly upregulated genes (>=1 log 2FoldChange) in exoRNeasy 2D and 3D conditions. FIG. 7C is a graph showing upset plot of unique and overlapping significantly downregulated genes (<=−1 log 2FoldChange) in exoRNeasy 2D and 3D conditions. FIG. 7D is a graph showing significantly enriched hallmark gene sets in exoRNeasy 3D with gene sets occurring in both 2D conditions highlighted in gray.

### DETAILED DESCRIPTION

KRAS is one of the most frequently mutated oncogenes in lung adenocarcinoma (LUAD). Non-invasive, predictive biomarkers for KRASG12C mutation-specific LUAD, however, remain largely unexplored. To date, there is no predictive relationship between lung cancer-secreted exosomal RNA and cancer severity, in particular for KRASG12C LUAD. As described herein, the extracellular RNA (exRNA) signatures were characterized of active and inhibited KRASG12C in lung cancer cells using exosome and extracellular vesicle (EV) isolation methods and RNA sequencing (RNA-seq). A comprehensive landscape of KRASG12C-regulated exRNA is described and 20 differentially expressed genes were identified that significantly associate with unfavorable clinical outcome based on The Cancer Genome Atlas (TCGA) LUAD data. This application provides mutant KRASG12C LUAD prognostic exRNA signatures that may serve as mutation- and tissue-specific biomarkers for non-invasive RNA liquid biopsies. A comprehensive atlas of KRASG12C-specific exRNA is described that can serve as a resource for RNA liquid biopsy-based detection development.

As used herein, the term “RAS pathway mutation” refers to a genetic mutation in a RAS pathway gene. Optionally, the RAS pathway mutation comprises a mutation in KRAS, NRAS, HRAS, EGFR, NF1, MET or BRAF. Optionally, the mutation is in KRAS. As used herein, the term “KRAS mutation” refers to a genetic mutation in the KRAS gene, which acts as an on-off switch in cell signaling and controls cell proliferation.

Provided herein are methods for detecting a RAS pathway mutation in a subject. The method includes obtaining a biological sample from the subject, isolating nucleic acids from the biological sample, and analyzing the expression level of extracellular RNAs in the nucleic acids, wherein a differential expression level of the extracellular RNAs compared to a control sample indicates that the subject has a RAS pathway mutation. The steps can be repeated one or more times. Optionally, the RAS pathway mutation is in KRAS. The KRAS mutation can be KRAS (G12C). The biological sample can comprise extracellular vesicles isolated from biofluids from the subject and the nucleic acids can comprise polyadenylated RNAs. Biofluids can be blood or serum. The subject can have or is suspected of having cancer. The cancer can be a RAS mutant cancer, e.g., a lung cancer. Optionally, isolating the nucleic acids comprises isolating extracellular vesicles from the biological sample followed by isolating the nucleic acids from the extracellular vesicles. The nucleic acids can be extracellular RNAs from the extracellular vesicles. Optionally, analyzing the expression of the extracellular RNAs comprises analyzing the expression of BNIP3, NUSAP1, OCIAD2, KRT18, ENO1, GAPDH, LDHA, UBE2S, CDKN3, KPNA2, ARHGAP11A, CENPF, ANLN, TPX2, HMMR, CCNB1, MAD2L1, BIRC5, GINS2, and UBE2C.

As described herein, the extracellular vesicles can comprise exosomes and/or microvesicles. Optionally, the extracellular vesicles are greater than 200 nm in size. Optionally, the extracellular vesicles are less than 200 nm in size.

The method can include an additional step of administering to the subject one or more anticancer agents. Optionally, the anticancer agent is an inhibitor of KRAS. The inhibitor of KRAS can be a small molecule, a nucleic acid or an antibody. Optionally, the small molecule is selected from the group consisting of MRTX-849, ARS1620, and AMG 510.

Analyzing expression levels can be carried out using any number of means including, but not limited to, polymerase chain reaction (PCR), reverse transcriptase polymerase chain reaction (RT-PCR), single-cell RNA-sequencing, microarray analysis, a Northern blot, serial analysis of gene expression (SAGE), immunoassay, hybridization capture, cDNA sequencing, direct RNA sequencing, nanopore sequencing, mass spectrometry, a CRISPR based technology, or combinations thereof. Optionally, analyzing the expression level of the extracellular RNAs comprises performing sequencing. The sequencing can comprise obtaining one or more sequencing reads of the extracellular RNAs and the analyzing can comprise aligning the sequencing reads of the extracellular RNAs to repetitive sequences in a human genome.

As noted above, the herein provided methods can further include administering to the subject one or more anticancer agents. The anticancer agent can be an inhibitor of KRAS. The method of analyzing the expression of extracellular RNAs can be repeated one or more times after administration of the anticancer agent.

As used herein, the term “polynucleotide” refers to an oligonucleotide, or nucleotide, and fragments or portions thereof, and to DNA or RNA of genomic or synthetic origin, which may be single- or double-stranded, and represent the sense or anti-sense strand. A single polynucleotide can be translated into a single polypeptide unless it is noncoding.

As used herein, the terms “peptide” and “polypeptide” are used interchangeably and describe a single polymer in which the monomers are amino acid residues which are joined together through amide bonds. A polypeptide is intended to encompass any amino acid sequence, either naturally occurring, recombinant, or synthetically produced.

As used herein, the term “substantial identity” or “substantially identical,” used in the context of nucleic acids or polypeptides, refers to a sequence that has at least 50% sequence identity with a reference sequence. Alternatively, percent identity can be any integer from 50% to 100%. In some embodiments, a sequence is substantially identical to a reference sequence if the sequence has at least 50%, 55%, 60%, 65%, 70%, 75%, 80%, 85%, 90%, 91%, 92%, 93%, 94%, 95%, 96%, 97%, 98%, or 99% sequence identity to the reference sequence as determined using, e.g., BLAST.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A comparison window includes reference to a segment of any one of the number of contiguous positions, e.g., a segment of at least 10 residues. In some embodiments, the comparison window has from 10 to 600 residues, e.g., about 10 to about 30 residues, about 10 to about 20 residues, about 50 to about 200 residues, or about 100 to about 150 residues, in which a sequence may be compared to a reference sequence of the same number of contiguous positions after the two sequences are optimally aligned.

Algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 25:3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference sequence if the smallest sum probability in a comparison of the test amino acid sequence to the reference amino acid sequence is less than about 0.01, more preferably less than about 10⁻⁵, and most preferably less than about 10⁻²⁰.

In the provided methods, if the gene in the biological sample from the subject displays a differential expression level relative to the corresponding reference gene in the control sample from the control subject, i.e., higher or lower than the expression level of the gene in the control sample by at least 2%, 4%, 6%, 8%, 10%, 20%, 30%, 40%, or 50%, then the subject may have cancer and/or a KRAS mutation. In certain embodiments, the cancer and/or the KRAS mutation may be in a tissue of the subject (e.g., lung).

In the methods described herein, an increased expression level of an extracellular RNA in a biological sample from a subject compared to a corresponding reference expression level of the same gene in a control sample from a control subject may indicate that the subject has cancer. Once it is determined that a subject (e.g., a subject suspected of having cancer) has an increased expression level of the gene relative to a control sample, the subject may be administered a therapeutically effective amount of an inhibitor to inhibit the expression level of the gene. In the methods described herein, the cancer can be a lung cancer (e.g., lung adenocarcinoma). The cancer may be characterized by an oncogenic defect in the RAS pathway. In particular, the oncogenic defect comprises an activating mutation in KRAS.

An inhibitor of the gene refers to an agent that inhibits or decreases the expression level and/or the activity of the gene. An inhibitor may inhibits or decreases the transcription of the gene, binds to the gene, and/or inhibits interaction between the gene and another protein or nucleic acid. In some embodiments, an inhibitor may be an inhibitory RNA (e.g., small interfering RNA (siRNA), an antisense RNA, microRNA (miRNA), and short hairpin RNA (shRNA)), an aptamer, an antibody, a CRISPR RNA or a small molecule.

An inhibitor may be an inhibitory RNA, e.g., small interfering RNA (siRNA), an antisense RNA, microRNA (miRNA), a CRISPR RNA or short hairpin RNA (shRNA). In some embodiments, the inhibitory RNA targets a sequence that is identical or substantially identical (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical) to a target sequence in the gene. A target sequence in the gene may be a portion of the gene comprising at least 20, at least 30, at least 40, at least 50, at least 60, at least 70, at least 80, at least 90, or at least 100 contiguous nucleotides, e.g., from 20-500, 20-250, 20-100, 50-500, or 50-250 contiguous nucleotides.

In the methods described herein, once it is determined that a subject (e.g., a subject suspected of having cancer) has an increased expression level of one or more noncoding RNAs relative to a control sample, the subject may be administered a therapeutically effective amount of an siRNA that inhibits or decreases the expression level of the gene. An siRNA may be produced from a short hairpin RNA (shRNA). A shRNA is an artificial RNA molecule with a hairpin turn that can be used to silence target gene expression via the siRNA it produces in cells. See, e.g., Fire et. al., Nature 391:806-811, 1998; Elbashir et al., Nature 411:494-498, 2001; Chakraborty et al., Mol Ther Nucleic Acids 8:132-143, 2017; and Bouard et al., Br. J. Pharmacol. 157:153-165, 2009. Expression of shRNA in cells is typically accomplished by delivery of plasmids or through viral or bacterial vectors. Suitable bacterial vectors include but not limited to adeno-associated viruses (AAVs), adenoviruses, and lentiviruses. After the vector has integrated into the host genome, the shRNA is then transcribed in the nucleus by polymerase II or polymerase III (depending on the promoter used). The resulting pre-shRNA is exported from the nucleus, then processed by Dicer and loaded into the RNA-induced silencing complex (RISC). The sense strand is degraded by RISC and the antisense strand directs RISC to an mRNA that has a complementary sequence. A protein called Ago2 in the RISC then cleaves the mRNA, or in some cases, represses translation of the mRNA, leading to its destruction and an eventual reduction in the protein encoded by the mRNA. Thus, the shRNA leads to targeted gene silencing.

Once it is determined that a subject (e.g., a subject suspected of having cancer) has an increased expression level of one or more noncoding RNAs relative to a control sample, the subject may be administered a therapeutically effective amount of an shRNA capable of hybridizing to a portion of the gene. The shRNA may be encoded in a vector. In some embodiments, the vector further comprises appropriate expression control elements known in the art, including, e.g., promoters (e.g., inducible promoters or tissue specific promoters), enhancers, and transcription terminators.

Once it is determined that a subject (e.g., a subject suspected of having cancer) has an increased expression level of one or more genes in described herein relative to a control sample, the subject may be administered a therapeutically effective amount of an siRNA capable of hybridizing to a portion of the gene. The siRNA may be encoded in a vector. In some embodiments, the vector further comprises appropriate expression control elements known in the art, including, e.g., promoters (e.g., inducible promoters or tissue specific promoters), enhancers, and transcription terminators.

In methods described herein, a subject may be administered one or more anticancer agents alone or in combination with one or more inhibitors that inhibit the expression levels of one or more noncoding RNAs. An anticancer agent may be a RAS pathway inhibitor, a cytotoxic agent, a chemotherapeutic agent, or an immunosuppressive agent. An anticancer agent may be a natural or synthetic agent. In some embodiments, an anticancer agent may be capable of treating cancer, activating immune response, and/or reducing tumor load. In some embodiments, an anticancer agent may inhibit the proliferation of and/or kill cancer cells. An anticancer agent may be a small molecule, a peptide, or a protein. In some embodiments, an anticancer agent may be an agent that inhibits and/or down regulates the activity of a protein that prevents immune cell activation or a protein that exerts immunosuppressive effects.

Examples of anticancer agents include, but are not limited to, RAS pathway inhibitors such as the mutant KRAS specific inhibitors including Sotorasib/AMG 510 (LUMARKRAS™), Adagrasib (MRTX849), MRTX1133, and GDC-6036; alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegall (see, e.g., Nicolaou et al. Angew. Chem Intl. Ed. Engl., 33:183-186 (1994)); CDP323, an oral alpha-4 integrin inhibitor; dynemicin, including dynemicin A; an esperamicin; neocarzinostatin chromophore and related chromoprotein enediyne antibiotic chromophores), aclacinomysins, actinomycin, authramycin, azaserine, bleomycin, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); combretastatin; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, 5-azacytidine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®, Bristol-Myers Squibb Oncology, Princeton, N.J.), albumin-engineered nanoparticle formulation of paclitaxel (ABRAXANE™), and docetaxel (TAXOTERER, Rhome-Poulene Rorer, Antony, France); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R) (e.g., erlotinib (Tarceva™)); and VEGF-A that reduce cell proliferation; vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g. celecoxib or etoricoxib), proteosome inhibitor (e.g. PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors; tyrosine kinase inhibitors; serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

An anticancer agent can be cisplatin, carboplatin, oxaliplatin, bleomycin, mitomycin C, calicheamicins, maytansinoids, doxorubicin, idarubicin, daunorubicin, epirubicin, busulfan, carmustine, lomustine, semustine, methotrexate, 6-mercaptopurine, fludarabine, 5-azacytidine, pentostatin, cytarabine, gemcitabine, 5-fluorouracil, hydroxyurea, etoposide, teniposide, topotecan, irinotecan, chlorambucil, cyclophosphamide, ifosfamide, melphalan, bortezomib, vincristine, vinblastine, vinorelbine, paclitaxel, or docetaxel.

The anticancer agent is a chemotherapeutic agent. In some embodiments, chemotherapeutic agents may kill cancer cells or inhibit cancer cell growth. Chemotherapeutic agents may function in a non-specific manner, for example, inhibiting the process of cell division known as mitosis. Examples of chemotherapeutic agents include, but are not limited to, antimicrotubule agents (e.g., taxanes and vinca alkaloids), topoisomerase inhibitors and antimetabolites (e.g., nucleoside analogs acting as such, for example, Gemcitabine), mitotic inhibitors, alkylating agents, antimetabolites, antitumor antibiotics, mitotic inhibitors, anthracyclines, intercalating agents, agents capable of interfering with a signal transduction pathway, agents that promote apoptosis, proteosome inhibitors, and alike.

Alkylating agents are most active in the resting phase of the cell. These types of drugs are cell-cycle non-specific. Exemplary alkylating agents include, but are not limited to, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNUR); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Antitumor antibiotics are chemotherapeutic agents obtained from natural products produced by species of the soil fungus, e.g., Streptomyces. These drugs act during multiple phases of the cell cycle and are considered cell-cycle specific. There are several types of antitumor antibiotics, including but are not limited to anthracyclines (e.g., Doxorubicin, Daunorubicin, Epirubicin, Mitoxantrone, and Idarubicin), chromomycins (e.g., Dactinomycin and Plicamycin), mitomycin, and bleomycin.

Antimetabolites are types of chemotherapeutic agents that are cell-cycle specific. When cells incorporate these antimetabolite substances into the cellular metabolism, they are unable to divide. This class of chemotherapeutic agents include folic acid antagonists such as Methotrexate; pyrimidine antagonists such as 5-Fluorouracil, Foxuridine, Cytarabine, Capecitabine, and Gemcitabine; purine antagonists such as 6-Mercaptopurine and 6-Thioguanine; Adenosine deaminase inhibitors such as Cladribine, Fludarabine, Nelarabine and Pentostatin.

Exemplary anthracyclines that can be used include, e.g., doxorubicin (Adriamycin® and Rubex®); Bleomycin (Lenoxane®); Daunorubicin (dauorubicin hydrochloride, daunomycin, and rubidomycin hydrochloride, Cerubidine®); Daunorubicin liposomal (daunorubicin citrate liposome, DaunoXome®); Mitoxantrone (DHAD, Novantrone®); Epirubicin (Ellence); Idarubicin (Idamycin®, Idamycin PFS®); Mitomycin C (Mutamycin®); Geldanamycin; Herbimycin; Ravidomycin; and Desacetylravidomycin.

Antimicrotubule agents include vinca alkaloids and taxanes. Exemplary vinca alkaloids include, but are not limited to, vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®). Exemplary taxanes that can be used include, but are not limited to paclitaxel and docetaxel. Non-limiting examples of paclitaxel agents include nanoparticle albumin-bound paclitaxel (ABRAXANE, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Bioorganic & Medicinal Chemistry Letters (2007) 17:617-620).

Exemplary proteosome inhibitors that can be used include, but are not limited to, Bortezomib (Velcade®); Carfilzomib (PX-171-007, (S)-4-Methyl-N—((S)-1-(((S)-4-methyl-1-((R)-2-methyloxiran-2-yl)-1-oxope-ntan-2-yl)amino)-1-oxo-3-phenylpropan-2-yl)-2-((S)-2-(2-morpholinoacetamid-o)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(-2R)-2-methyl-2-oxiranyl]-2-oxo-1-(phenylmethyl)ethyl]-L-serinamide (ONX-0912).

The chemotherapeutic agent can be chlorambucil, cyclophosphamide, ifosfamide, melphalan, streptozocin, carmustine, lomustine, bendamustine, uramustine, estramustine, carmustine, nimustine, ranimustine, mannosulfan busulfan, dacarbazine, temozolomide, thiotepa, altretamine, 5-fluorouracil (5-FU), 6-mercaptopurine (6-MP), capecitabine, cytarabine, floxuridine, fludarabine, gemcitabine, hydroxyurea, methotrexate, pemetrexed, daunorubicin, doxorubicin, epirubicin, idarubicin, SN-38, ARC, NPC, campothecin, topotecan, 9-nitrocamptothecin, 9-aminocamptothecin, rubifen, gimatecan, diflomotecan, BN80927, DX-895 If, MAG-CPT, amsacrine, etoposide, etoposide phosphate, teniposide, doxorubicin, paclitaxel, docetaxel, gemcitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, gemcitabine, Irinotecan, albumin-bound paclitaxel, Oxaliplatin, Capecitabine, Cisplatin, docetaxel, irinotecan liposome, and etoposide, and combinations thereof.

The chemotherapeutic agent can be administered at a dose and a schedule that may be guided by doses and schedules approved by the U.S. Food and Drug Administration (FDA) or other regulatory body, subject to empirical optimization. Optionally, more than one chemotherapeutic agent may be administered simultaneously, or sequentially in any order during the entire or portions of the treatment period. The two agents may be administered following the same or different dosing regimens.

Techniques and methods for measuring the expression levels of genes are available in the art. For example, detection and/or quantification of noncoding RNAs may be accomplished by any one of a number methods or assays employing recombinant DNA or RNA technologies known in the art, including but not limited to, polymerase chain reaction (PCR), single-cell RNA-sequencing, reverse transcription PCR (RT-PCR), microarrays, Northern blot, serial analysis of gene expression (SAGE), immunoassay, hybridization capture, cDNA sequencing, direct RNA sequencing, nanopore sequencing, CRISPR based technology, and mass spectrometry.

Hybridization capture methods may be used for detection and/or quantification of the noncoding RNAs. Some examples of hybridization capture methods include, e.g., capture hybridization analysis of RNA targets (CHART), chromatin isolation by RNA purification (ChIRP), CRISPR based technology and RNA affinity purification (RAP). In general, cells and tissues expressing the RNA of interest can be cross-linked and solubilized by shearing. The RNA of interest can then be enriched using rationally designed biotin tagged antisense oligonucleotides. The captured RNA complexes can then be rinsed and eluted. The eluted material can be analyzed for the molecules of interest. The associated RNAs are commonly analyzed with qPCR or high throughput sequencing, and the recovered proteins can be analyzed with Western blots or mass spectrometry. General techniques for performing hybridization capture methods are described in the art and can be found in, e.g., Machyna and Simon, Briefings in Functional Genomics 17 (2): 96-103, 2018, which is incorporated herein by reference in its entirety. Further, Li et al, JCI Insight. 3 (7): e98942, 2018 also describes methods of studying RNA (e.g., extracellular RNA) and is incorporated herein by reference in its entirety.

Microarrays may be used to measure the expression levels of the genes. An advantage of microarray analysis is that the expression of each of the genes can be measured simultaneously, and microarrays can be specifically designed to provide a diagnostic expression profile for a particular disease or condition (e.g., cancer). Microarrays may be prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic nucleic acids. The polynucleotide sequences of the probes may also be synthesized nucleotide sequences, such as synthetic oligonucleotide sequences. Probes may be immobilized to a solid support which may be either porous or non-porous. For example, the probes may be polynucleotide sequences which are attached to a nitrocellulose or nylon membrane or filter covalently at either the 3′ or the 5′ end of the polynucleotide. Such hybridization probes are well-known in the art (see, e.g., Sambrook, et al., Molecular Cloning: A Laboratory Manual (3rd Ed., 2001). In one embodiment, a microarray may include a support or surface with an ordered array of binding (e.g., hybridization) sites or “probes” each representing one of the genes described herein. More specifically, each probe of the array may be located at a known, predetermined position on the solid support such that the identity (i.e., the sequence) of each probe can be determined from its position in the array (i.e., on the support or surface). Each probe may be covalently attached to the solid support at a single site.

Quantitative reverse transcriptase PCR (qRT-PCR) can also be used to determine the expression profiles of the genes. The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMY-RT) and Moloney murine leukemia virus reverse transcriptase (MLVRT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, CA, USA), following the manufacturer's instructions. The derived cDNA can then be used as a template in the subsequent PCR reaction. Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TAQMAN PCR typically utilizes the 5′-nuclease activity of Taq polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, may be designed to detect nucleotide sequence located between the two PCR primers. The probe is non-extendible by Taq DNA polymerase enzyme, and may be labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

Serial Analysis Gene Expression (SAGE) can also be used to determine RNA expression level. SAGE analysis does not require a special device for detection, and may be used for simultaneously detecting the expression of a large number of transcription products. First, RNA is extracted, converted into cDNA using a biotinylated oligo (dT) primer, and treated with a four-base recognizing restriction enzyme (Anchoring Enzyme: AE) resulting in AE-treated fragments containing a biotin group at their 3′ terminus. Next, the AE-treated fragments are incubated with streptavidin for binding. The bound cDNA is divided into two fractions, and each fraction is then linked to a different double-stranded oligonucleotide adapter (linker) A or B. These linkers are composed of: (1) a protruding single strand portion having a sequence complementary to the sequence of the protruding portion formed by the action of the anchoring enzyme, (2) a 5′ nucleotide recognizing sequence of the IIS-type restriction enzyme (cleaves at a predetermined location no more than 20 bp away from the recognition site) serving as a tagging enzyme (TE), and (3) an additional sequence of sufficient length for constructing a PCR-specific primer. The linker-linked cDNA is cleaved using the tagging enzyme, and only the linker-linked cDNA sequence portion remains, which is present in the form of a short-strand sequence tag. Next, pools of short-strand sequence tags from the two different types of linkers are linked to each other, followed by PCR amplification using primers specific to linkers A and B. As a result, the amplification product is obtained as a mixture comprising myriad sequences of two adjacent sequence tags (ditags) bound to linkers A and B. The amplification product is treated with the anchoring enzyme, and the free ditag portions are linked into strands in a standard linkage reaction. The amplification product is then cloned. Determination of the clone's nucleotide sequence can be used to obtain a readout of consecutive ditags of constant length. The presence of the gene corresponding to each tag can then be identified from the nucleotide sequence of the clone and information on the sequence tags.

### EXAMPLE

### Example 1. Exosomal RNA Signatures of KRASG12C Lung Adenocarcinomas

The RAS family of proteins—HRAS, NRAS, and KRAS—are highly conserved in eukaryotes and enable signal transduction that mediates, in addition to a plethora of other functions, cellular proliferation¹. This central role in enabling the expansion of cellular populations lends itself to malignant co-option: 19% of global cancer patients harbor a mutant KRAS allele². The fraction of lung adenocarcinoma (LUAD) tumors with a mutant KRAS allele is even higher: estimates from The Cancer Genome Atlas (TCGA) suggest roughly 30% of patients have detectable KRAS mutations^{3,4}. Moreover, approximately 85% of these occur at codons 12, 13, or 61³. Polymorphisms at Glycine 12 in KRAS lock this GTPase in its active, GTP-bound state that enables constitutive and oncogenic signaling¹. Long thought to be ‘undruggable’, KRASG12C has recently been targeted successfully, and specifically, by the small molecule inhibitors MRTX-849, ARS1620, and AMG 510⁵⁻⁷. These inhibitors are not only life-saving clinical treatments but also tools that will help advance the understanding of KRASG12C-dependent phenotypes, outcomes, and transcriptional programs^{5,6,8}.

While effective KRASG12C inhibition has been confirmed for AMG 510, MRTX-849, and ARS1620, longitudinal analysis of each has demonstrated that abundance of KRAS and its downstream effector, phosphorylated ERK (PERK), begin to recover at 24 hours of AMG 510 and ARS1620 treatment in NCI-H358 cells^{6,7,9}. In particular, by 72 hours of AMG 510 treatment, NCI-H358 cells exhibit a marked shift in transcriptional programming that has been implicated as a ‘resistance’ mechanism that co-opts PI3K signaling to induce epithelial-to-mesenchymal transition (EMT)¹⁰.

For inhibitors like AMG 510 to have maximal impact on the patient population, we need a more robust methodology to identify and characterize KRASG12C-driven cancers. A path towards more accessible cancer diagnostics is being charted by non-invasive, RNA- or DNA-based liquid biopsies that rely on transcriptional signatures or genomic methylation, respectively¹¹⁻¹⁴. Recent advances have confirmed the presence and detectability of long RNAs as both freely available cell-free RNAs and RNA encapsulated within extracellular vesicles (EVs) secreted from tissues^{11,15-17}. Extracellular RNAs (exRNAs) packaged within EVs are protected from degradation and are sufficiently complex to detect protein-coding, long noncoding, mitochondrial, and ribosomal RNA^{15,16}. Isolation of the EVs from human blood plasma or serum generally requires one of three approaches: ultra-centrifugation, which is slow and cumbersome^{15,16}, affinity column extraction, which is accurate and effective but limited to certain classes of EV¹⁸, and microfluidic filtration, which is fast, modular, dependent only on size of the EV particles, but not widely adopted^{19,20.}

Moreover, prevailing evidence suggests that some exRNAs are dependent on intracellular events and reflect intracellular transcriptional paradigms in developmental and tumorigenic contexts^{21,22}. These exRNAs appear to be involved in intracellular communication and trans-regulation of gene expression²³. These cell-derived signatures are robust enough to deconvolve into specific cell types of origin when captured in vivo²⁴. KRAS in particular appears to have strong regulatory influence over exRNA secretion and identity, making it a promising context for exRNA biomarker discovery^{17,25,26}.

In this study, we utilize both affinity column and microfluidic EV isolation to generate exRNA sequencing libraries from cell culture media of the LUAD-derived H358 cell line (KRAS^{G12C}/Tp53^{-/-}). We demonstrate the utility of the AMG 510 KRASG12C inhibitor to identify KRASG12C-specific transcriptional signatures and the importance of EV extraction methodology to optimize RNA liquid biopsy performance. The transcriptional signatures derived from our in vitro model demonstrate strong agreement with KRASG12C-positive tumor samples from the TCGA LUAD dataset. Our work demonstrates the potential of exRNA signatures of KRASG12C for developing a non-invasive, RNA liquid biopsy companion diagnostic to identify LUAD patients that would benefit from AMG 510 targeted therapy.

### Results

AMG 510 inhibition of KRAS^{G12C} in lung cancer cells.

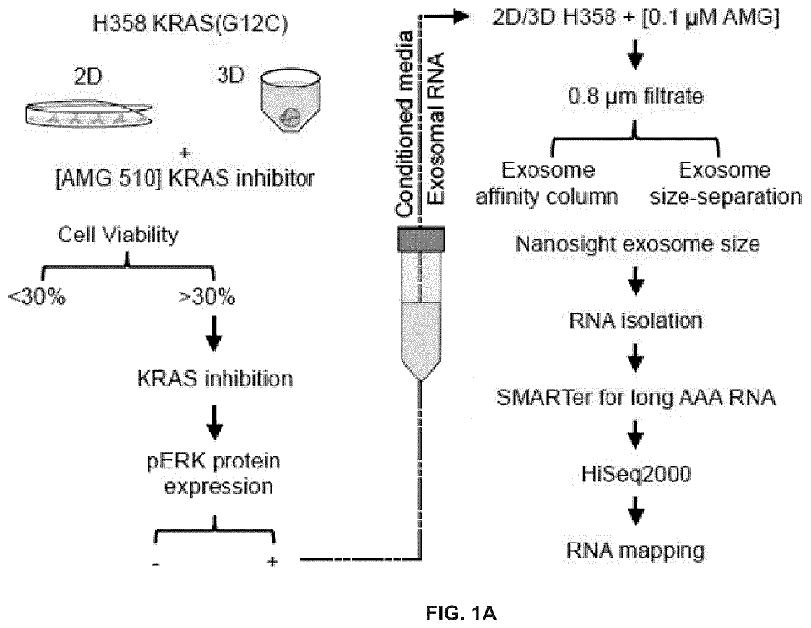

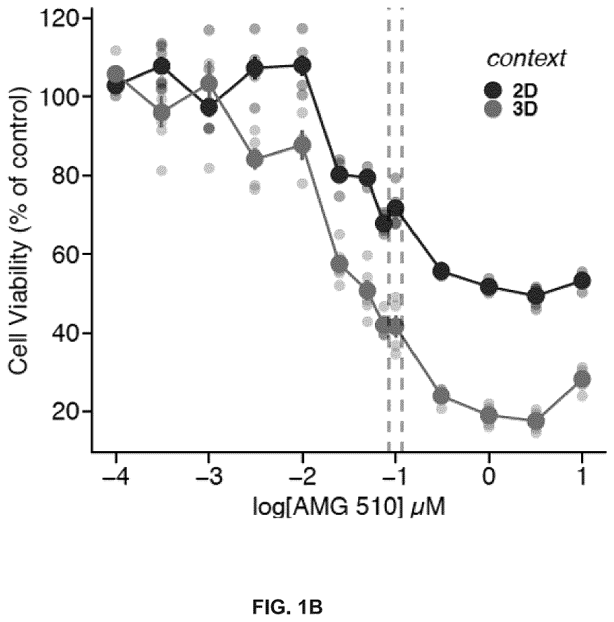

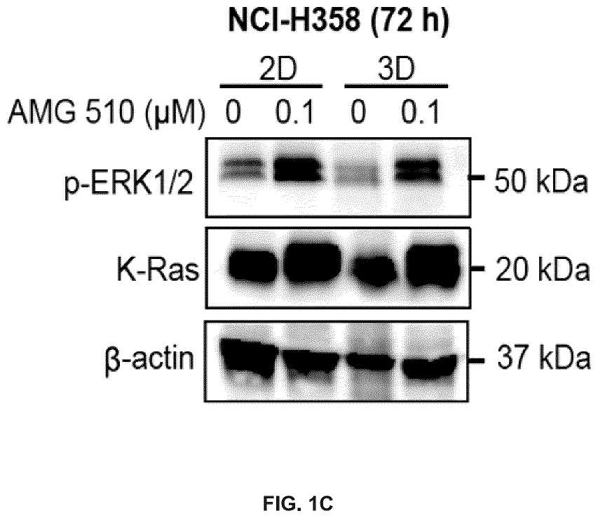

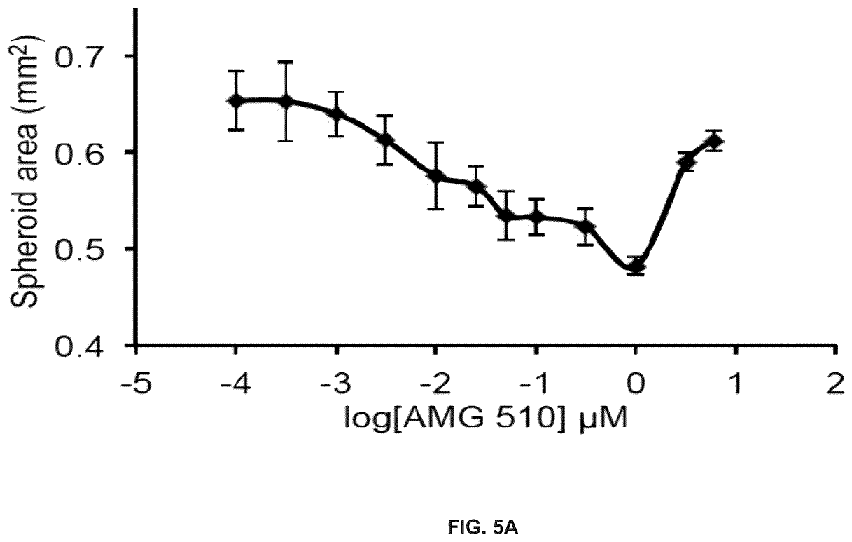

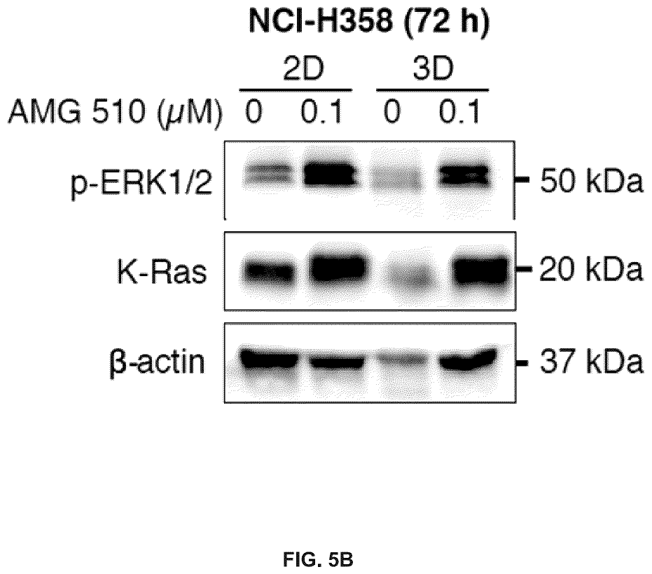

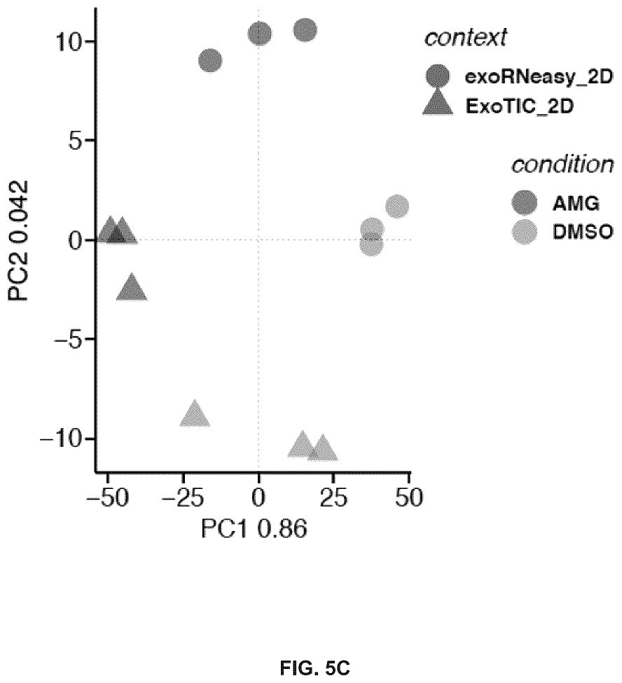

To investigate the effects of active and inhibited KRAS^{G12C} on the exRNA profiles in EVs secreted by the H358 cell line, we first determined the optimal concentration of AMG 510 inhibitor (IC₅₀) while maintaining sufficiently viable cells to produce EVs (FIG. 1A). A range of AMG 510 concentrations from 10⁻⁴ to 10 μM, with matched DMSO controls, added in two-dimensional (2D) adherent monolayer and three-dimensional (3D) spheroid cultures of KRAS^{G12C} H358 lung cancer cells resulted in IC₅₀ values of 0.0465 μM and 0.025 μM, respectively, after 72 hours of treatment (FIG. 1B). 2D and 3D cell cultures retained 71% and 41% viability compared to controls, respectively, after treatment with 0.1 μM of AMG 510. The size of 3D spheroids treated with 0.1 μM AMG 510 decreased 20% with respect to control conditions. However, consistent with the cell viability data, higher AMG 510 concentrations of 10 μM had less effect on spheroid sizes (FIG. 5C). Western blots against phosphorylated ERK (pERK) and KRAS were performed to verify inhibition of KRAS^{G12C} signaling and demonstrated an increase in AMG 510-bound KRAS, although pERK was not effectively suppressed (FIG. 1C).

exRNAs from lung cancer cell EVs change in response to KRAS^{G12C} inhibition.

To characterize exRNA profiles, EVs were isolated using two orthogonal approaches: affinity column extraction (ExoRNeasy) and size-based filtration (Exosome Total Isolation Chip: ExoTIC). Each EV isolation method captured distinct EV populations as determined by Nanoparticle Tracking Analysis (NTA) (FIGS. 5A and 5B) 18.19. The ExoTIC platform captured a low yield of EVs that fell within a size range matching that of exosomes (FIG. 5A). The size profile of EVs shifted to a smaller distribution, around 150 nm, after AMG 510 treatment. While ExoRNeasy appeared to capture a higher yield of EVs, their size profile was centered above 200 nm, suggesting that a different class of EVs were being captured, such as microvesicles. ExoRNeasy also captured a smaller EV size range upon AMG 510 treatment (FIG. 5B)

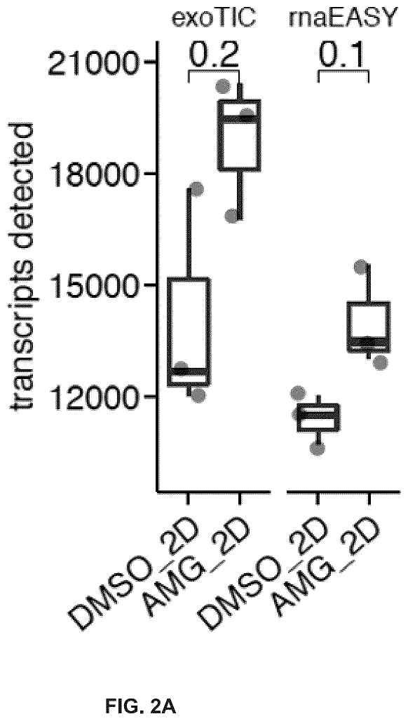

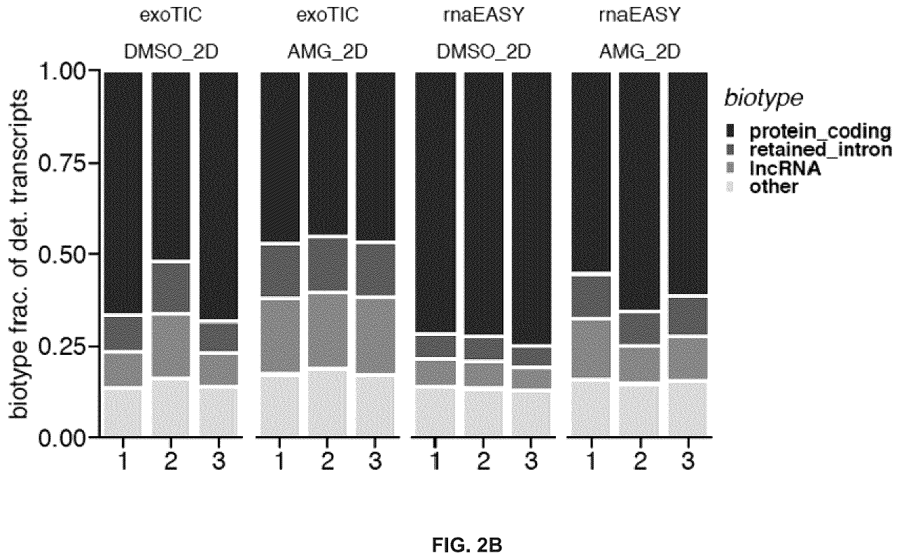

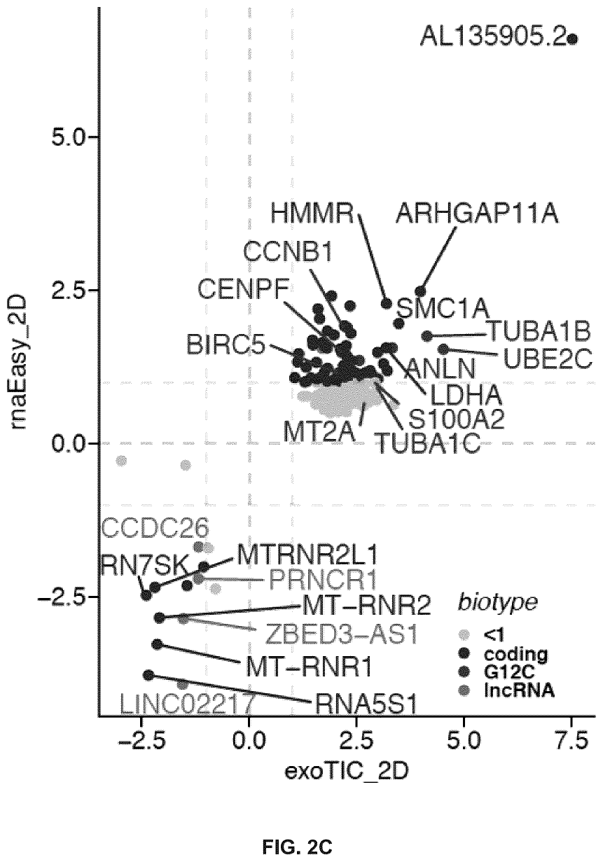

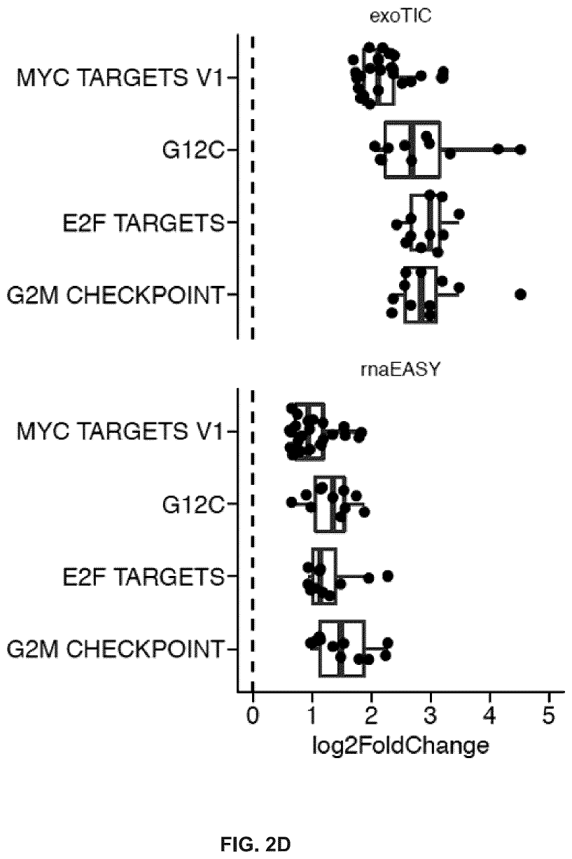

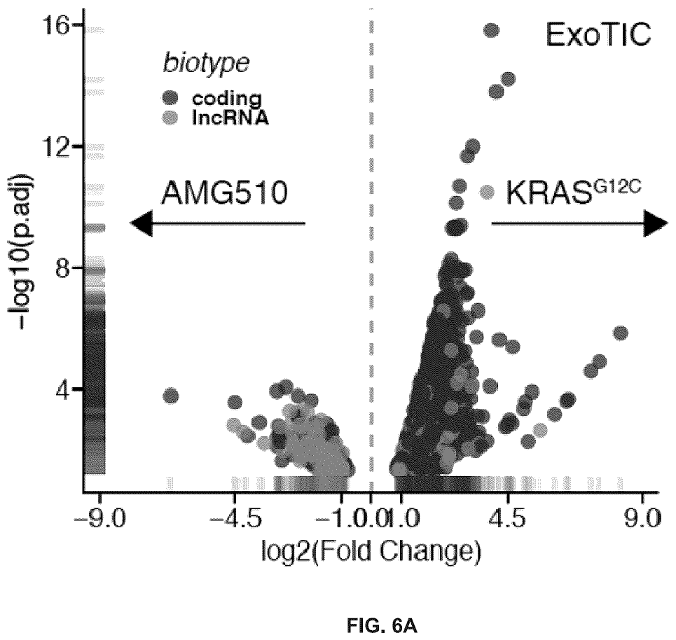

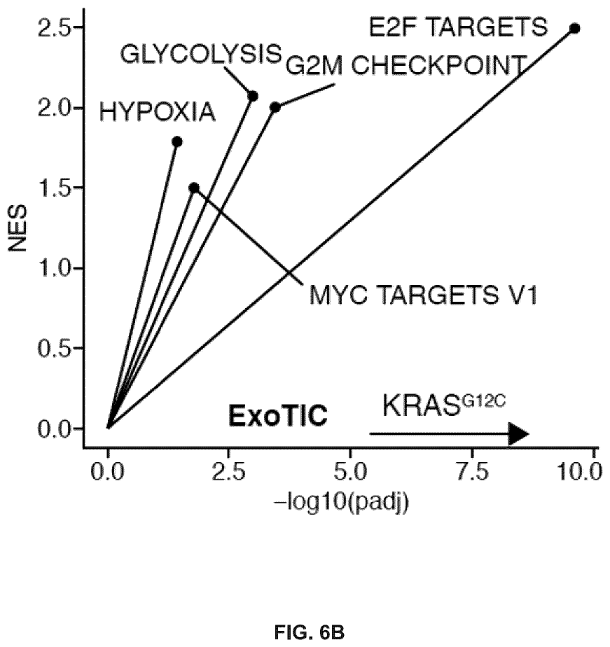

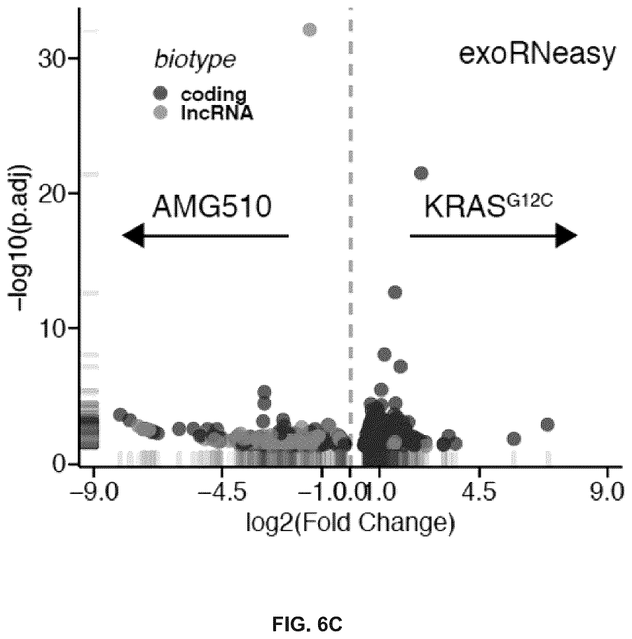

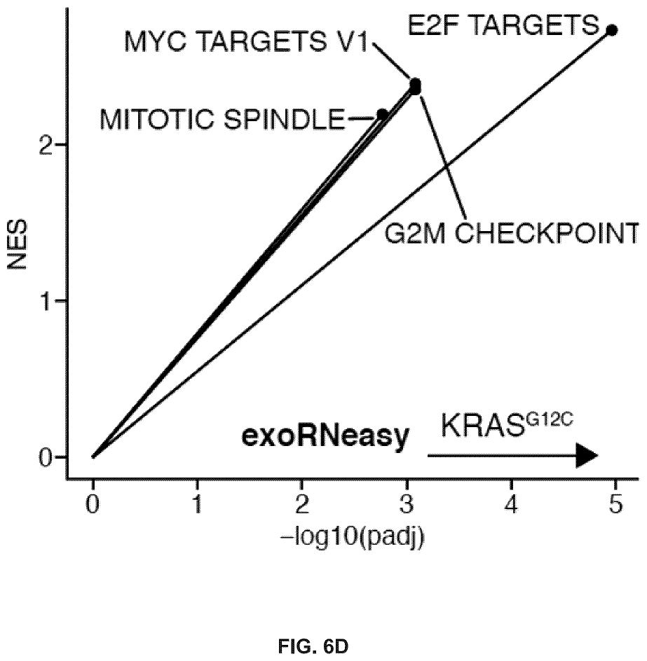

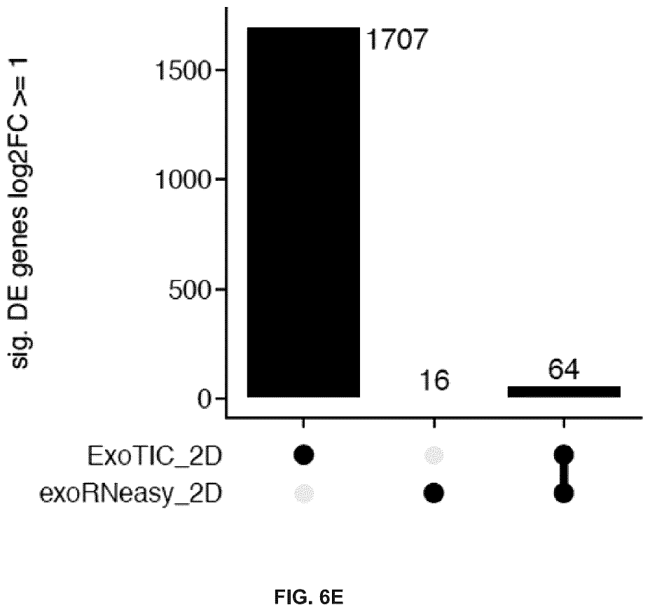

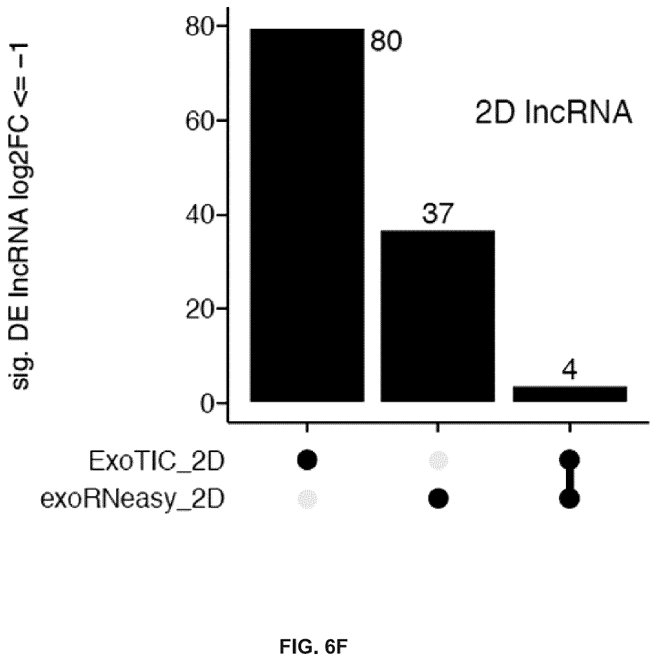

RNA sequencing (RNA-seq) libraries prepared from EVs extracted by size-based ExoTIC demonstrated greater transcriptional complexity and variability than the equivalent using ExoRNeasy. Both platforms exhibited no significant alterations in exRNA complexity upon AMG 510 treatment (FIG. 2A). RNA-seq captured primarily protein-coding transcripts, although this majority was significantly decreased in ExoTIC-derived exRNAs, which demonstrated an increased abundance of lncRNAs, retained introns, and other noncoding RNA biotypes (FIG. 2B). Despite these differences, the two EV isolation platforms had modest agreement in differentially expressed (DE) genes between DMSO (positive log change) and AMG (negative log change) conditions (FIG. 2C, 6E), with 64 shared significantly upregulated genes (padj <=0.05, log 2FoldChange >=1). Nine of these genes were determined to be induced by KRAS^{G12C} by Xue et al (FIG. 2C) 9. Furthermore, the two platforms demonstrated strong agreement in Gene Set Enrichment Analysis (GSEA), with overlapping enrichment of 3 hallmark gene sets provided by MSigDB: MYC TARGETS V1, E2F TARGETS, and G2M CHECKPOINT (FIG. 2D, FIGS. 6B and 6D)²⁷. While the overlapping DE genes represented the majority of all DE genes in the ExoRNeasy comparison (64/80), they represented a minority of the DE genes observed in ExoTIC data (64/1771) (FIG. 6E).

KRAS^{G12C} inhibition leads to enrichment of lncRNA, mtRNA, and TE RNA in EVs.

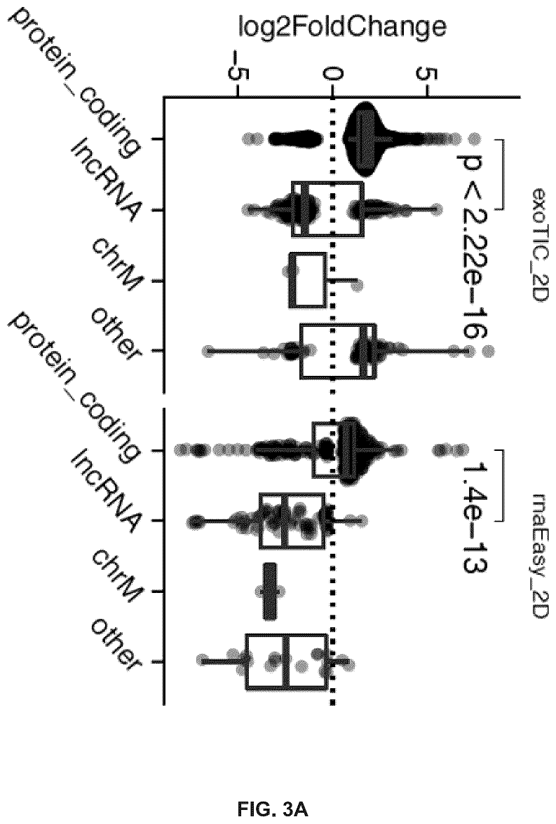

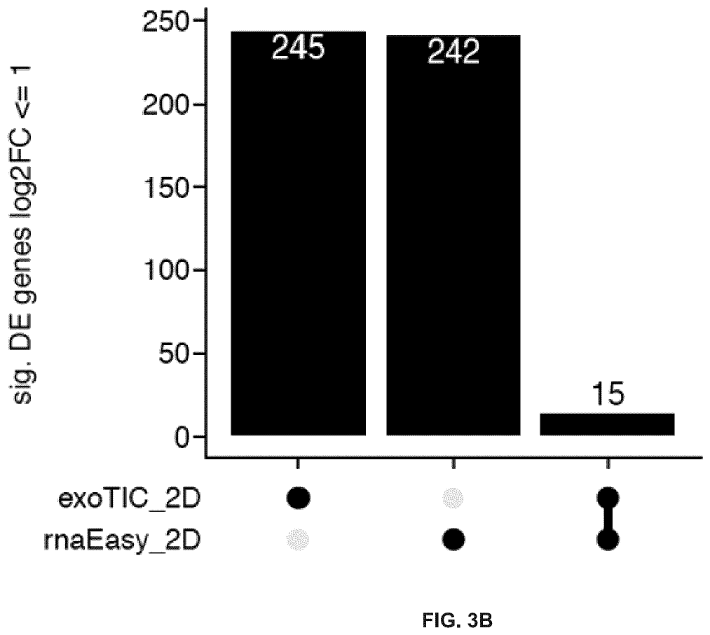

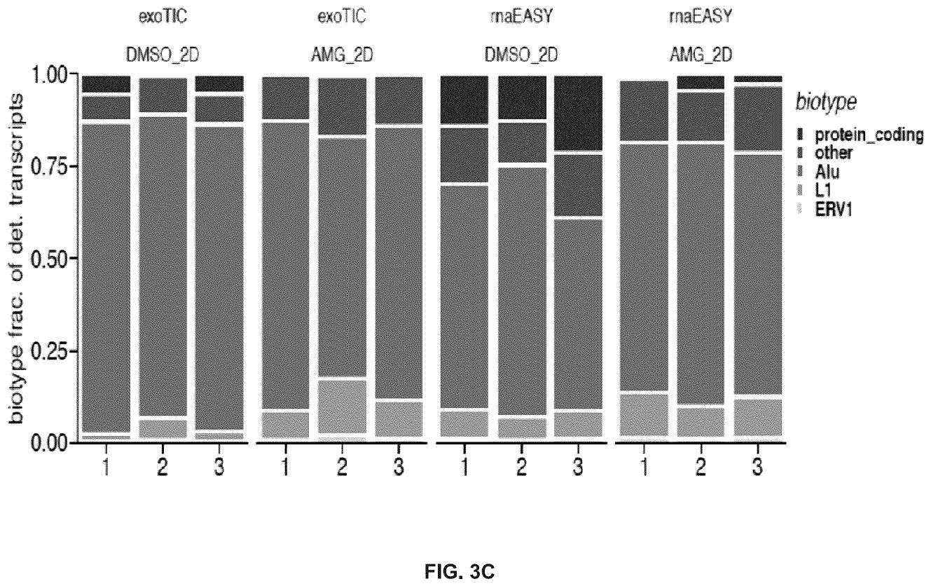

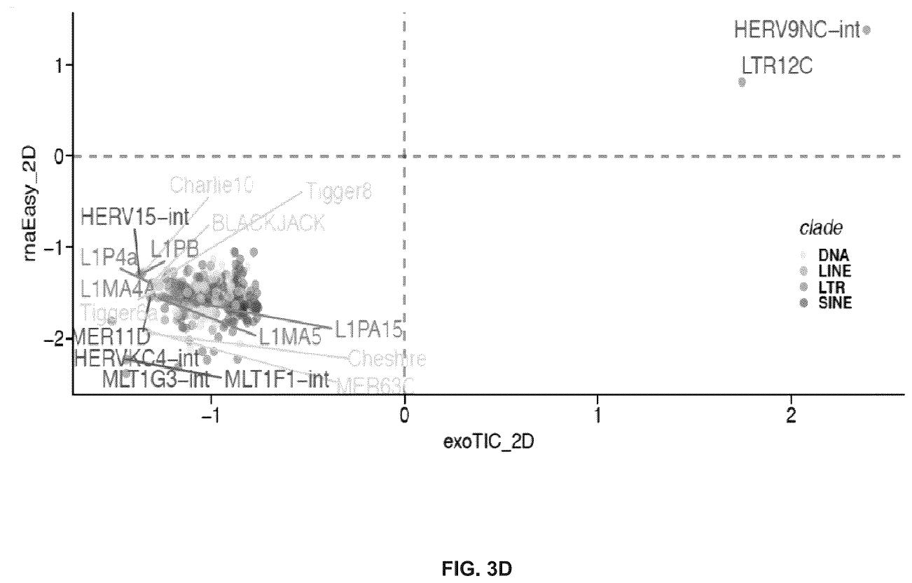

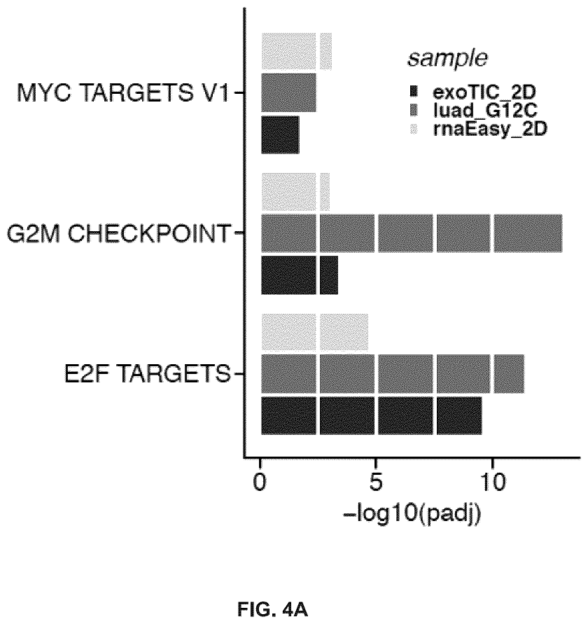

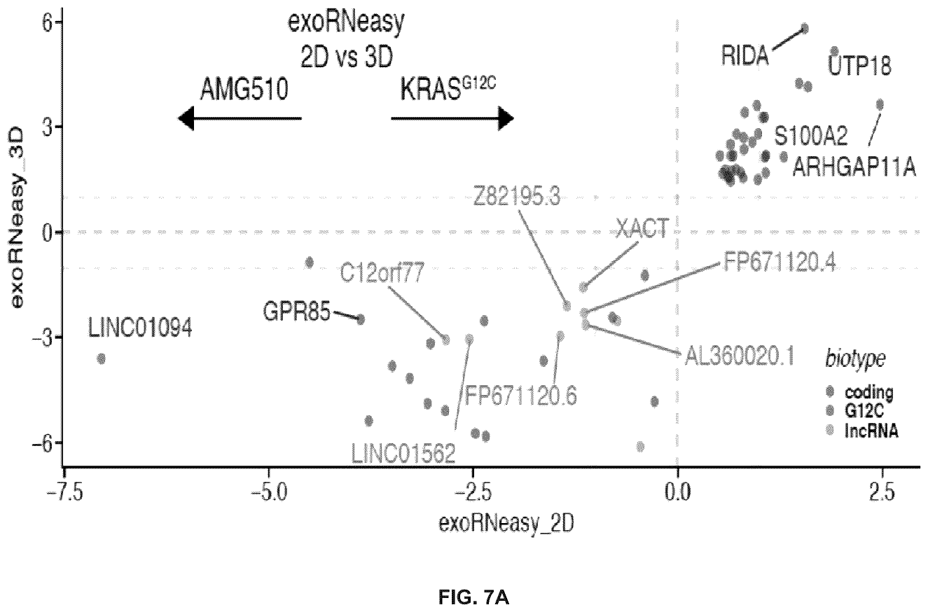

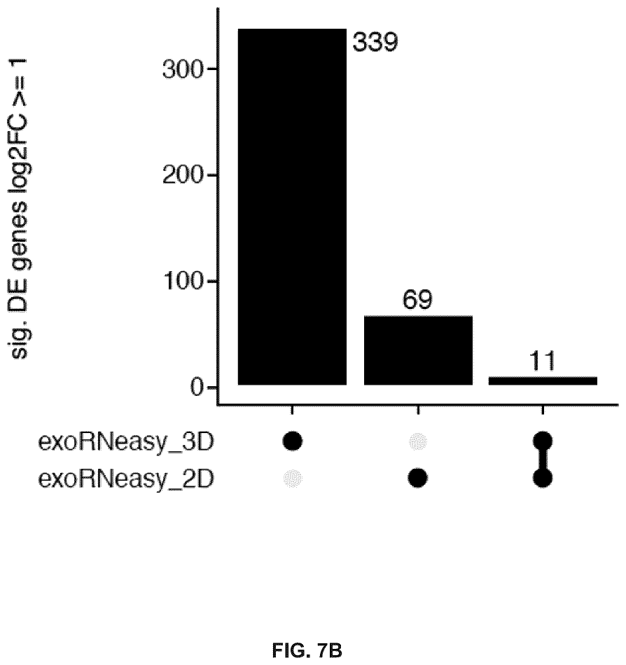

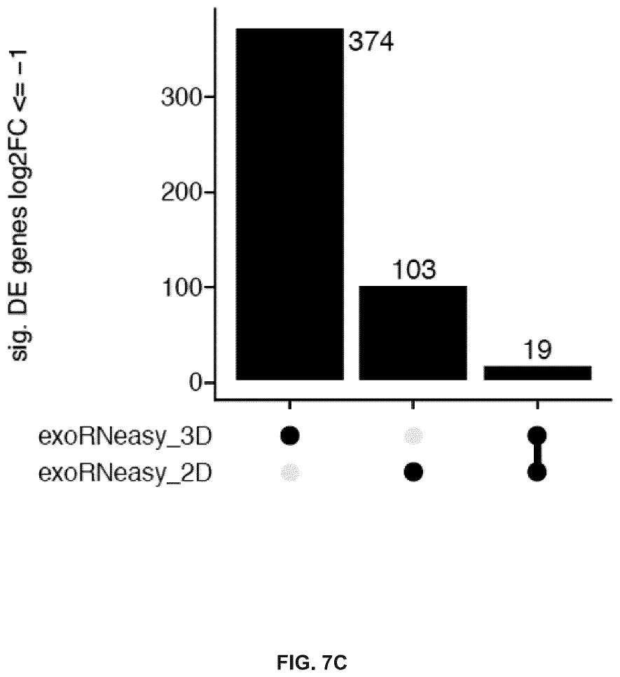

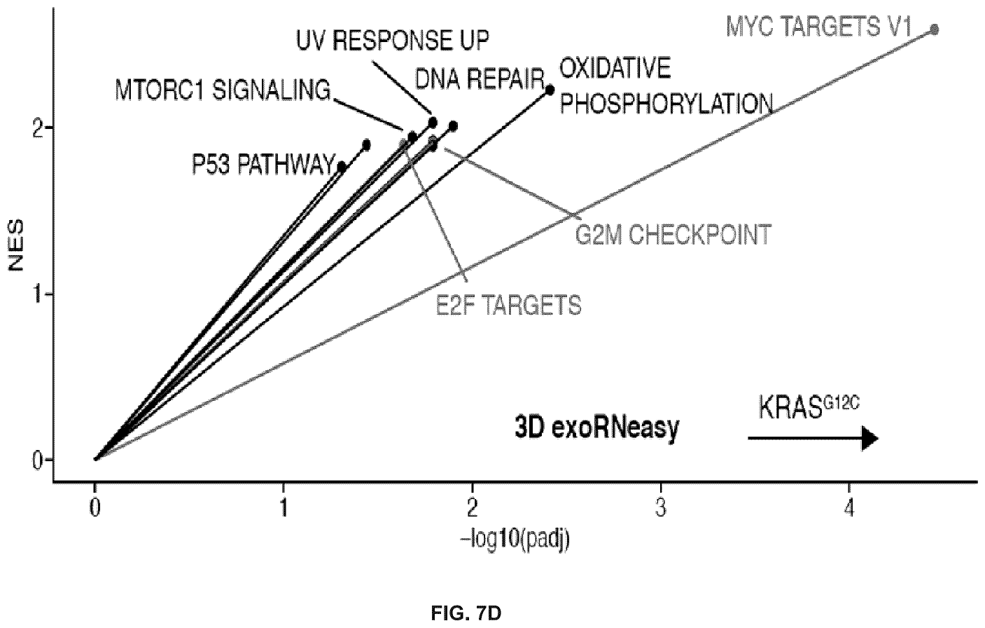

We next examined the effect of AMG 510 KRAS^{G12C} inhibition on exRNA identity and abundance. exRNA-seq captured RNA biotypes that are selectively enriched by AMG 510 treatment (negative log 2FoldChange, FIG. 2C). In particular, there was a significant enrichment in negatively DE lncRNA genes (bimodal) across both platforms (FIG. 3A), with only 5 genes that overlapped (FIG. 7B). This reflected a global shift in comparison to that of the upregulated fraction of DE genes: each platform detected hundreds of uniquely downregulated genes (FIG. 3B). In addition to lncRNAs, mitochondrial-encoded RNAs (mtRNA), particularly ribosomal (mt-rRNA), were strongly biased towards enrichment in the inhibited context (FIG. 1A).

The remaining biotype enriched in the exRNAs from AMG 510-treated cells was Transposable Element (TE) RNA, which are among the most abundantly expressed transcripts in the human genome²⁸. The dysregulation of TEs, canonically silenced in somatic tissues, has been observed in numerous cancers^{29,30}. Mutant KRAS appears to directly affect regulatory programs known to control TE expression²⁶. In order to determine the dynamics of TE expression in the context of KRAS^{G12C} inhibition, we included TE insertions in our quantification reference for RNA-seq analysis. The addition of TE sequences revealed that TE RNA was the most abundant aligned biotype (FIG. 3C). This predominance was only exacerbated by KRAS^{G12C} inhibition, which elicited a strong enrichment in the majority of DE TE-derived RNAs across both platforms (FIG. 3D).

exRNA signatures of KRAS^{G12C} activity discriminate LUAD from healthy lung tissue.

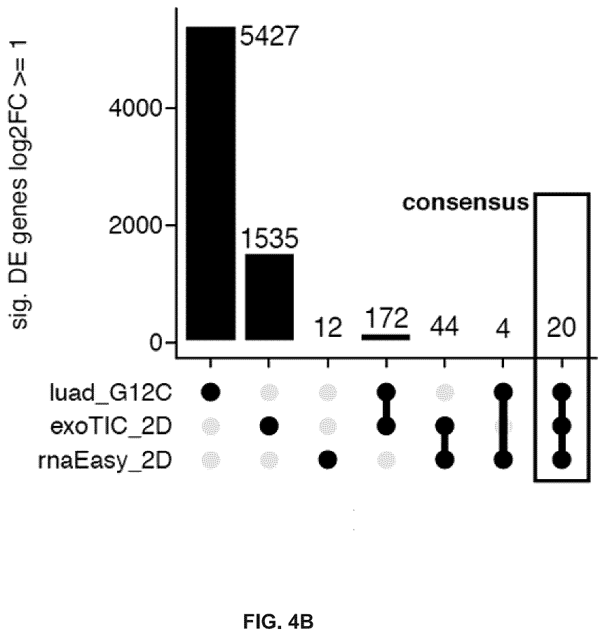

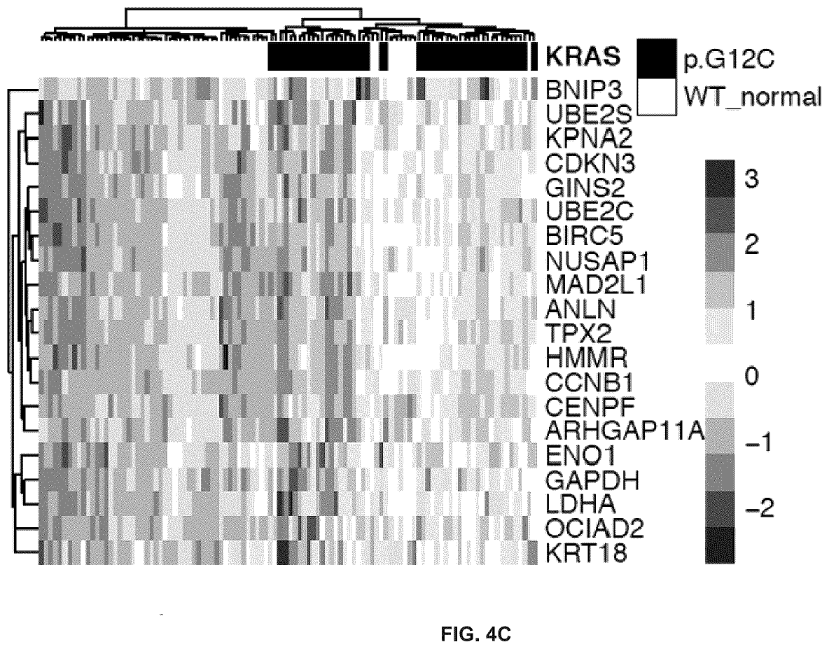

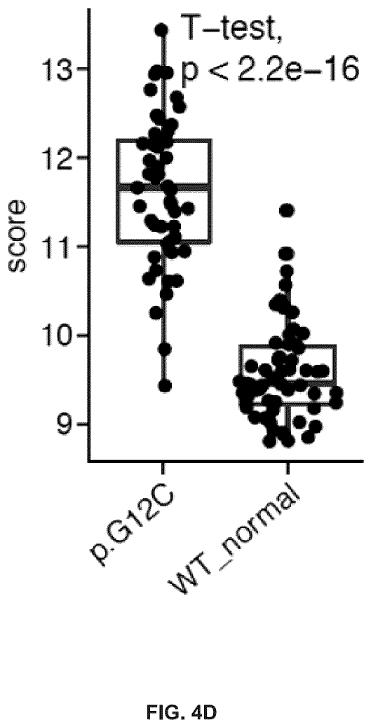

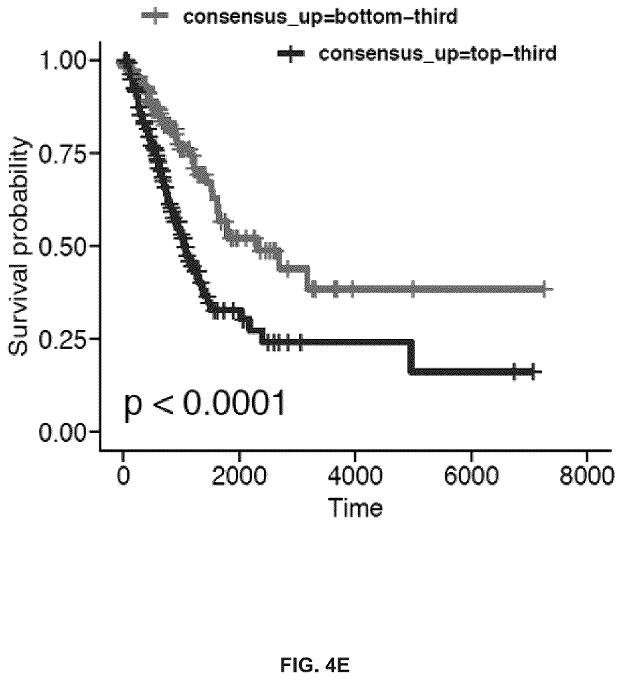

Lastly, to determine the relevance of the exRNA signatures detected in both the ExoTIC and ExoRNeasy platforms, we utilized RNA-seq counts from the TCGA LUAD cohort. Initial DE and GSEA approaches identified shared enrichment of 3 hallmark gene sets (MYC TARGETS V1, E2F TARGETS, and G2M CHECKPOINT) and a consensus upregulation of 20 genes across the in vitro and in vivo datasets (FIG. 4A,B). Table 1 lists the consensus gene signature and Table 2 lists all genes. Hierarchical clustering of the LUAD cohort using this 20-gene signature produced robust separation between the G12C LUAD tumor samples and the healthy (WT) lung tissue samples (FIG. 4C). Furthermore, the average per-sample expression of this consensus signature was used to represent a patient ‘score’ for G12C activity that produced significantly different distributions in G12C and WT samples, respectively (FIG. 4D). Finally, this per-sample average was used to stratify the LUAD cohort into thirds, from which the top-third highest expressing and bottom-third lowest expressing were extracted and compared in a Kaplan-Meier survival analysis, revealing a significant decrease in survival probability for the top-third expressing samples (FIG. 4E).

TABLE 1
Consensus 20 Gene Signature of KRAS Mutant Cells.

| | ExoTIC_{—} | exoRNeasy_{—} | luad_TCGA_{—} | ExoTIC_{—} | exoRNeasy_{—} | luad_TCGA_{—} |
|---|---|---|---|---|---|---|
| gene | padi | padj | padj | log2FC | log2FC | log2FC |
| ANLN | 1.20E−04 | 0.017090909 | 1.69E−60 | 3.330394872 | 1.556448639 | 3.838710922 |
| ARHGAP11A | 3.65E−18 | 9.54E−23 | 5.73E−35 | 3.986223297 | 2.476901011 | 1.925707455 |
| BIRC5 | 5.75E−06 | 0.004062783 | 3.42E−59 | 2.059731408 | 1.143083642 | 3.386641002 |
| BNIP3 | 1.88E−05 | 0.003393005 | 1.41E−29 | 2.257641334 | 1.593765657 | 1.451683155 |
| CCNB1 | 1.56E−04 | 0.005180157 | 2.76E−75 | 2.287550875 | 1.885577172 | 2.899571528 |
| CDKN3 | 3.37E−07 | 0.003711706 | 4.88E−36 | 2.990430203 | 1.485601686 | 2.681221462 |
| CENPF | 5.04E−05 | 0.004062783 | 1.98E−83 | 2.152654263 | 1.485681587 | 3.343683562 |
| ENO1 | 1.64E−09 | 5.17E−06 | 3.84E−26 | 2.483911206 | 1.075388988 | 1.164513461 |
| GAPDH | 3.47E−12 | 1.13E−08 | 2.93E−47 | 2.816107718 | 1.185363978 | 1.808098742 |
| GINS2 | 1.47E−05 | 0.040491381 | 2.51E−52 | 2.564058205 | 1.35175989 | 2.635649747 |
| HMMR | 0.003555909 | 0.039654102 | 1.81E−51 | 3.197707595 | 2.278683607 | 2.896725819 |
| KPNA2 | 1.55E−07 | 0.002672587 | 4.27E−42 | 2.843062987 | 1.142338344 | 1.825572638 |
| KRT18 | 1.23E−07 | 6.82E−05 | 1.23E−25 | 2.28979415 | 1.080954581 | 1.579190106 |
| LDHA | 1.15E−13 | 9.60E−14 | 5.30E−29 | 3.188569621 | 1.557578424 | 1.65484667 |
| MAD2L1 | 0.003425178 | 0.001337101 | 8.12E−41 | 1.820101353 | 1.832347551 | 2.181911014 |
| NUSAP1 | 0.027808475 | 0.020468152 | 1.62E−50 | 1.475972829 | 1.684005384 | 2.381663548 |
| OCIAD2 | 1.11E−04 | 0.001539736 | 3.67E−73 | 1.647860724 | 1.06537927 | 2.246983325 |
| TPX2 | 0.004581138 | 0.020633061 | 4.60E−67 | 2.348403818 | 2.242565997 | 3.307165895 |
| UBE2C | 1.10E−15 | 3.34E−04 | 8.12E−66 | 4.522017497 | 1.534187551 | 4.145517644 |
| UBE2S | 9.22E−06 | 0.019578016 | 1.23E−32 | 2.585145031 | 1.124178818 | 1.810425659 |

| gene | ensg | chr(hg38) | start | end | width | strand |
|---|---|---|---|---|---|---|
| ANLN | ENSG00000011426.11 | chr7 | 36389821 | 36453791 | 63971 | + |
| ARHGAP11A | ENSG00000198826.11 | chr15 | 32615144 | 32639941 | 24798 | + |
| BIRC5 | ENSG00000089685.15 | chr17 | 78214186 | 78225636 | 11451 | + |
| BNIP3 | ENSG00000176171.11 | chr10 | 131966455 | 131982013 | 15559 | − |
| CCNB1 | ENSG00000134057.15 | chr5 | 69167135 | 69178245 | 11111 | + |
| CDKN3 | ENSG00000100526.20 | chr14 | 54396849 | 54420218 | 23370 | + |
| CENPF | ENSG00000117724.13 | chr1 | 214603195 | 214664571 | 61377 | + |
| ENO1 | ENSG00000074800.16 | chr1 | 8861000 | 8879190 | 18191 | − |
| GAPDH | ENSG00000111640.15 | chr12 | 6534512 | 6538374 | 3863 | + |
| GINS2 | ENSG00000131153.9 | chr16 | 85676198 | 85690073 | 13876 | − |
| HMMR | ENSG00000072571.20 | chr5 | 163460203 | 163491941 | 31739 | + |
| KPNA2 | ENSG00000182481.9 | chr17 | 68035708 | 68046854 | 11147 | + |
| KRT18 | ENSG00000111057.11 | chr12 | 52948871 | 52952906 | 4036 | + |
| LDHA | ENSG00000134333.14 | chr11 | 18394560 | 18408425 | 13866 | + |
| MAD2L1 | ENSG00000164109.14 | chr4 | 120055623 | 120066858 | 11236 | − |
| NUSAP1 | ENSG00000137804.14 | chr15 | 41320794 | 41381050 | 60257 | + |
| OCIAD2 | ENSG00000145247.12 | chr4 | 48885019 | 48906937 | 21919 | − |
| TPX2 | ENSG00000088325.16 | chr20 | 31739271 | 31801805 | 62535 | + |
| UBE2C | ENSG00000175063.17 | chr20 | 45812576 | 45816957 | 4382 | + |
| UBE2S | ENSG00000108106.14 | chr19 | 55399745 | 55407788 | 8044 | − |

TABLE 2
Extracellular RNA Signatures of KRAS Mutant Cells.

| | ExoTIC_{—} | exoRNeasy_{—} | ExoTIC_{—} | exoRNeasy_{—} | |
|---|---|---|---|---|---|
| gene | padj | padj | log2FC | log2FC | ensg |
| AL135905.2 | 1.85E−05 | 0.006852609 | 7.523034323 | 6.591156232 | ENSG00000285976.2 |
| UBE2C | 1.10E−15 | 3.34E−04 | 4.522017497 | 1.534187551 | ENSG00000175063.17 |
| TUBA1B | 1.10E−15 | 2.05E−07 | 4.140251553 | 1.74850414 | ENSG00000123416.15 |
| ARHGAP11A | 3.65E−18 | 9.54E−23 | 3.986223297 | 2.476901011 | ENSG00000198826.11 |
| SMC1A | 1.98E−06 | 0.003711706 | 3.483683028 | 1.956493607 | ENSG00000072501.19 |
| LGALS1 | 7.15E−14 | 0.022316796 | 3.370215639 | 0.637467345 | ENSG00000100097.12 |
| ANLN | 1.20E−04 | 0.017090909 | 3.330394872 | 1.556448639 | ENSG00000011426.11 |
| NOP56 | 2.51E−07 | 0.005380667 | 3.216921834 | 1.185135518 | ENSG00000101361.17 |
| HMMR | 0.003555909 | 0.039654102 | 3.197707595 | 2.278683607 | ENSG00000072571.20 |
| LDHA | 1.15E−13 | 9.60E−14 | 3.188569621 | 1.557578424 | ENSG00000134333.14 |
| HMGB2 | 3.34E−09 | 0.003575263 | 3.125673673 | 1.299069011 | ENSG00000164104.12 |
| CKS2 | 2.30E−09 | 0.005401958 | 2.993645305 | 1.054640773 | ENSG00000123975.5 |
| CDKN3 | 3.37E−07 | 0.003711706 | 2.990430203 | 1.485601686 | ENSG00000100526.20 |
| TUBA1C | 1.77E−08 | 0.043973363 | 2.983713673 | 0.902178698 | ENSG00000167553.16 |
| RPL21 | 2.68E−11 | 0.004110939 | 2.969343076 | 0.706432986 | ENSG00000122026.10 |
| S100A2 | 8.27E−13 | 3.34E−04 | 2.929645007 | 0.983483981 | ENSG00000196754.13 |
| KPNA2 | 1.55E−07 | 0.002672587 | 2.843062987 | 1.142338344 | ENSG00000182481.9 |
| ACTB | 3.84E−11 | 0.003575263 | 2.840455693 | 0.773393636 | ENSG00000075624.17 |
| GAPDH | 3.47E−12 | 1.13E−08 | 2.816107718 | 1.185363978 | ENSG00000111640.15 |
| ANXA2 | 3.09E−11 | 0.007955737 | 2.780752738 | 0.675609826 | ENSG00000182718.17 |
| S100A10 | 2.68E−11 | 4.67E−04 | 2.758668191 | 0.805780934 | ENSG00000197747.9 |
| TUBB4B | 1.99E−06 | 0.012800314 | 2.705965319 | 1.155212949 | ENSG00000188229.6 |
| ALDOA | 3.84E−11 | 5.04E−04 | 2.684677526 | 0.976476853 | ENSG00000149925.22 |
| MT2A | 5.64E−10 | 0.033290401 | 2.682114008 | 0.654839034 | ENSG00000125148.7 |
| RAN | 1.77E−08 | 9.73E−04 | 2.671310684 | 0.931681791 | ENSG00000132341.12 |
| CKS1B | 1.21E−07 | 0.008734525 | 2.667557449 | 0.975891866 | ENSG00000173207.13 |
| TMSB4X | 3.90E−10 | 0.004384145 | 2.658174712 | 0.612084585 | ENSG00000205542.11 |
| UBE2S | 9.22E−06 | 0.019578016 | 2.585145031 | 1.124178818 | ENSG00000108106.14 |
| GINS2 | 1.47E−05 | 0.040491381 | 2.564058205 | 1.35175989 | ENSG00000131153.9 |
| RPL23A | 1.33E−09 | 0.005006575 | 2.560912253 | 0.646828157 | ENSG00000198242.14 |
| TAGLN2 | 7.89E−08 | 0.002164273 | 2.530731202 | 0.801401715 | ENSG00000158710.15 |
| RPS2P5 | 2.33E−08 | 0.021966139 | 2.530683663 | 0.866048057 | ENSG00000240342.3 |
| PGK1 | 1.92E−08 | 0.007614152 | 2.520334171 | 0.806539535 | ENSG00000102144.15 |
| CALM2 | 4.35E−09 | 0.013764293 | 2.500175963 | 0.680972811 | ENSG00000143933.19 |
| FABP5 | 9.98E−08 | 0.003711706 | 2.496052185 | 1.145228951 | ENSG00000164687.11 |
| ENO1 | 1.64E−09 | 5.17E−06 | 2.483911206 | 1.075388988 | ENSG00000074800.16 |
| ANP32E | 2.33E−08 | 0.017247737 | 2.429721213 | 0.935034663 | ENSG00000143401.15 |
| TXN | 7.89E−08 | 0.006952326 | 2.427646336 | 0.772149419 | ENSG00000136810.13 |
| SH3BGRL3 | 1.21E−07 | 0.023414394 | 2.426613972 | 0.810748059 | ENSG00000142669.15 |
| PGAM1 | 2.35E−07 | 0.001279486 | 2.423535076 | 1.169382783 | ENSG00000171314.9 |
| RPLP1 | 3.93E−08 | 1.79E−05 | 2.419431408 | 0.714133599 | ENSG00000137818.12 |
| RBM25 | 5.85E−08 | 7.92E−05 | 2.419004705 | 0.890883111 | ENSG00000119707.14 |
| GTF3A | 1.03E−06 | 0.019504698 | 2.417510257 | 0.970055754 | ENSG00000122034.16 |
| ESF1 | 1.64E−08 | 0.002196174 | 2.397730107 | 0.911208775 | ENSG00000089048.14 |
| SRSF7 | 1.68E−06 | 0.012800314 | 2.389275972 | 1.348526819 | ENSG00000115875.19 |
| TFDP1 | 5.76E−04 | 0.012800314 | 2.368595333 | 1.793286777 | ENSG00000198176.13 |
| RPS2 | 3.01E−08 | 0.00929189 | 2.368307465 | 0.644913626 | ENSG00000140988.16 |
| TPX2 | 0.004581138 | 0.020633061 | 2.348403818 | 2.242565997 | ENSG00000088325.16 |
| HNRNPA2B1 | 1.54E−06 | 0.038537122 | 2.342080381 | 0.75112859 | ENSG00000122566.21 |
| BOLA3 | 1.11E−06 | 9.73E−04 | 2.329836595 | 1.238796108 | ENSG00000163170.12 |
| UBE2N | 1.36E−06 | 0.046182356 | 2.317752375 | 0.924191105 | ENSG00000177889.10 |
| SNRPE | 1.23E−07 | 0.032072201 | 2.301023811 | 0.656703837 | ENSG00000182004.13 |
| RPS3 | 2.12E−08 | 0.012513576 | 2.29508954 | 0.629165593 | ENSG00000149273.15 |
| STMN1 | 1.87E−07 | 0.012898877 | 2.289815695 | 0.70489283 | ENSG00000117632.23 |
| KRT18 | 1.23E−07 | 6.82E−05 | 2.28979415 | 1.080954581 | ENSG00000111057.11 |
| UACA | 9.86E−07 | 0.020087351 | 2.288829084 | 0.722331211 | ENSG00000137831.15 |
| CCNB1 | 1.56E−04 | 0.005180157 | 2.287550875 | 1.885577172 | ENSG00000134057.15 |
| TMA7 | 2.72E−07 | 8.05E−04 | 2.277234507 | 0.912375077 | ENSG00000232112.3 |
| RPL41 | 5.82E−08 | 0.004110939 | 2.2768297 | 0.649465424 | ENSG00000229117.9 |
| METAP1 | 0.002032163 | 0.036275057 | 2.276769546 | 1.422403227 | ENSG00000164024.12 |
| S100A16 | 4.85E−07 | 0.00908002 | 2.271634686 | 0.942178673 | ENSG00000188643.11 |
| RPS25 | 1.87E−07 | 0.027288652 | 2.267182342 | 0.515026499 | ENSG00000118181.11 |
| BNIP3 | 1.88E−05 | 0.003393005 | 2.257641334 | 1.593765657 | ENSG00000176171.11 |
| RPSA | 1.69E−07 | 0.018736546 | 2.255350279 | 0.644398721 | ENSG00000168028.14 |
| RPL7 | 4.70E−08 | 0.001346038 | 2.24528601 | 0.671916158 | ENSG00000147604.14 |
| RPS27A | 1.18E−07 | 0.006852609 | 2.233524649 | 0.550394424 | ENSG00000143947.14 |
| UBE2V1 | 5.02E−06 | 0.006015704 | 2.228395058 | 1.080934044 | ENSG00000244687.12 |
| RPS20 | 6.88E−08 | 3.35E−04 | 2.21834196 | 0.770794823 | ENSG00000008988.10 |
| UTP18 | 0.002744624 | 0.02934113 | 2.217776276 | 1.916891556 | ENSG00000011260.14 |
| PCLAF | 5.70E−06 | 0.005380667 | 2.21301696 | 1.532661926 | ENSG00000166803.14 |
| VDAC1 | 4.01E−06 | 0.003491223 | 2.193231755 | 1.192363145 | ENSG00000213585.11 |
| SKA2 | 3.86E−06 | 0.013346109 | 2.190546736 | 1.266975369 | ENSG00000182628.13 |
| CCND1 | 6.33E−06 | 4.67E−04 | 2.180121286 | 1.17429142 | ENSG00000110092.4 |
| RPS18 | 1.21E−07 | 8.13E−04 | 2.170579363 | 0.714974185 | ENSG00000231500.7 |
| ANXA5 | 4.91E−05 | 0.020461885 | 2.170460953 | 1.033847314 | ENSG00000164111.15 |
| MRPL13 | 6.48E−06 | 0.033290401 | 2.166056949 | 0.911260818 | ENSG00000172172.8 |
| RPL12 | 1.71E−07 | 8.68E−05 | 2.164305267 | 0.743095333 | ENSG00000197958.13 |
| RPLP0 | 1.69E−07 | 8.12E−04 | 2.159881132 | 0.765091869 | ENSG00000089157.16 |
| CENPF | 5.04E−05 | 0.004062783 | 2.152654263 | 1.485681587 | ENSG00000117724.13 |
| RPL18A | 4.89E−07 | 0.02934113 | 2.134470472 | 0.594300662 | ENSG00000105640.13 |
| FTL | 9.68E−07 | 0.006299364 | 2.126715524 | 0.712009752 | ENSG00000087086.15 |
| PRELID1 | 6.92E−06 | 0.002835761 | 2.1257596 | 1.013079462 | ENSG00000169230.10 |
| UQCRC1 | 7.05E−05 | 0.028688548 | 2.121455192 | 0.798736808 | ENSG00000010256.11 |
| SNRPA1 | 1.26E−04 | 6.92E−04 | 2.121163051 | 1.538877066 | ENSG00000131876.17 |
| NME1 | 2.28E−06 | 0.007955737 | 2.118764815 | 0.959260603 | ENSG00000239672.8 |
| EEF1B2 | 5.17E−07 | 0.010897558 | 2.115410281 | 0.666335341 | ENSG00000114942.14 |
| RPL9 | 9.44E−08 | 0.004114748 | 2.095508838 | 0.649370927 | ENSG00000163682.17 |
| RPS17 | 1.23E−07 | 0.022222871 | 2.080964585 | 0.561303261 | ENSG00000182774.13 |
| PFN1 | 1.40E−06 | 0.036457026 | 2.079899985 | 0.648753079 | ENSG00000108518.8 |
| AC243919.1 | 6.19E−07 | 0.027288652 | 2.07278766 | 0.515715301 | ENSG00000237550.5 |
| RPL32 | 1.67E−06 | 0.004763095 | 2.067369145 | 0.717635026 | ENSG00000144713.13 |
| HMGB1 | 2.04E−06 | 0.004062783 | 2.067070375 | 0.834619311 | ENSG00000189403.15 |
| RPS3A | 1.23E−07 | 0.005763741 | 2.064420724 | 0.627239508 | ENSG00000145425.10 |
| NDUFS5 | 1.03E−06 | 0.026728344 | 2.063878315 | 0.646805299 | ENSG00000168653.11 |
| BIRC5 | 5.75E−06 | 0.004062783 | 2.059731408 | 1.143083642 | ENSG0000089685.15 |
| RPL35 | 8.61E−07 | 0.017090909 | 2.055355244 | 0.648882971 | ENSG00000136942.15 |
| RPL5 | 3.26E−07 | 0.036275057 | 2.05339435 | 0.581446429 | ENSG00000122406.14 |
| NME2 | 2.92E−06 | 7.92E−05 | 2.037904953 | 0.935055018 | ENSG00000243678.12 |
| KRT8 | 7.19E−06 | 9.73E−04 | 2.01830615 | 0.828460684 | ENSG00000170421.12 |
| ARPC1A | 2.31E−05 | 0.037310096 | 2.005062523 | 0.776947968 | ENSG00000241685.10 |
| PTMA | 1.74E−06 | 0.038364413 | 1.998202481 | 0.549936234 | ENSG00000187514.16 |
| RPS14 | 1.16E−06 | 0.021715718 | 1.997205092 | 0.573121554 | ENSG00000164587.13 |
| SNU13 | 2.74E−06 | 9.73E−04 | 1.984271877 | 1.037828507 | ENSG00000100138.15 |
| C1QBP | 3.60E−05 | 0.022115216 | 1.979976472 | 0.966957619 | ENSG00000108561.8 |
| HNRNPA1 | 1.88E−06 | 0.003575263 | 1.977689995 | 0.65553192 | ENSG00000135486.18 |
| LYAR | 7.37E−04 | 0.017090909 | 1.974023854 | 1.768859998 | ENSG00000145220.14 |
| RPS6 | 9.86E−07 | 0.009413403 | 1.965694032 | 0.670139799 | ENSG00000137154.13 |
| EIF5B | 8.69E−06 | 0.003711706 | 1.9588158 | 0.870199505 | ENSG00000158417.11 |
| PXMP2 | 0.023126542 | 0.023767014 | 1.918954991 | 2.404044079 | ENSG00000176894.10 |
| RPS7 | 4.74E−06 | 0.03400889 | 1.889087858 | 0.497752888 | ENSG00000171863.15 |
| CALM1 | 7.83E−06 | 0.010897558 | 1.886052518 | 0.784579874 | ENSG00000198668.13 |
| CNBP | 7.19E−06 | 0.03492985 | 1.87519119 | 0.777266418 | ENSG00000169714.17 |
| EIF5 | 2.11E−05 | 0.02452416 | 1.860507045 | 0.700110625 | ENSG00000100664.11 |
| ATP5ME | 9.27E−06 | 0.031709943 | 1.850770818 | 0.59351155 | ENSG00000169020.10 |
| PNN | 5.89E−05 | 0.048527126 | 1.846577305 | 0.981772868 | ENSG00000100941.9 |
| SNRPN | 1.04E−04 | 0.035226463 | 1.833521529 | 0.744570952 | ENSG00000128739.23 |
| HSBP1 | 8.67E−06 | 0.031709943 | 1.826552931 | 0.606780848 | ENSG00000230989.7 |
| GCSH | 3.37E−05 | 7.45E−04 | 1.82558725 | 1.227633449 | ENSG00000140905.11 |
| MAD2L1 | 0.003425178 | 0.001337101 | 1.820101353 | 1.832347551 | ENSG00000164109.14 |
| VDAC3 | 0.001829837 | 0.034767153 | 1.819056947 | 1.015669902 | ENSG00000078668.14 |
| RIDA | 0.001976561 | 0.02841888 | 1.81704079 | 1.552074433 | ENSG00000132541.11 |
| FTH1 | 1.92E−05 | 0.001765753 | 1.812640993 | 0.687393662 | ENSG00000167996.16 |
| SNHG16 | 1.43E−04 | 0.007789247 | 1.797852209 | 0.934302188 | ENSG00000163597.15 |
| HSPD1 | 6.08E−05 | 0.042054623 | 1.783374935 | 0.720561767 | ENSG00000144381.17 |
| MAL2 | 6.63E−05 | 0.044098372 | 1.769615784 | 0.649260848 | ENSG00000147676.14 |
| COTL1 | 1.80E−04 | 4.60E−05 | 1.768357575 | 1.577809172 | ENSG00000103187.8 |
| PA2G4 | 0.001461666 | 0.011211203 | 1.753813489 | 1.155730952 | ENSG00000170515.14 |
| HSP90AA1 | 2.60E−05 | 0.028587398 | 1.744363479 | 0.551264066 | ENSG00000080824.19 |
| RPL14 | 2.25E−05 | 0.029522831 | 1.734375196 | 0.618779581 | ENSG00000188846.14 |
| JPT1 | 5.51E−05 | 0.02934113 | 1.703501285 | 0.778741733 | ENSG00000189159.16 |
| CCT8 | 5.23E−04 | 5.04E−04 | 1.702026974 | 1.639242232 | ENSG00000156261.13 |
| AC125611.3 | 0.004126316 | 0.037183134 | 1.698167807 | 1.550477199 | ENSG00000258232.2 |
| CCT5 | 6.38E−05 | 0.022398385 | 1.695536914 | 0.820566872 | ENSG00000150753.12 |
| LDHB | 1.71E−04 | 0.02452416 | 1.693848212 | 0.643523988 | ENSG00000111716.14 |
| UBE2D3 | 2.31E−05 | 0.047451151 | 1.687310201 | 0.67601752 | ENSG00000109332.20 |
| RPS12 | 1.07E−04 | 0.001252625 | 1.663108809 | 0.800650126 | ENSG00000112306.8 |
| OCIAD2 | 1.11E−04 | 0.001539736 | 1.647860724 | 1.06537927 | ENSG00000145247.12 |
| SELENOF | 0.006926533 | 9.40E−04 | 1.644507549 | 2.032178673 | ENSG00000183291.17 |
| MRPL39 | 0.02581509 | 0.017477864 | 1.601208038 | 2.191173065 | ENSG00000154719.14 |
| UBE2V2 | 3.51E−04 | 0.005099195 | 1.581767861 | 1.048947117 | ENSG00000169139.12 |
| SPTSSA | 0.001895344 | 0.024089015 | 1.575075149 | 0.994720473 | ENSG00000165389.7 |
| DYNC1LI2 | 1.27E−04 | 0.005763741 | 1.569082659 | 0.778959288 | ENSG00000135720.13 |
| OSTC | 0.004026413 | 0.003711706 | 1.565693026 | 1.32290666 | ENSG00000198856.13 |
| PSMA6 | 1.30E−04 | 0.035226463 | 1.549146558 | 0.644108015 | ENSG00000100902.11 |
| NIFK | 0.003710408 | 8.73E−04 | 1.486868369 | 1.606601909 | ENSG00000155438.12 |
| NUSAP1 | 0.027808475 | 0.020468152 | 1.475972829 | 1.684005384 | ENSG00000137804.14 |
| YBX3 | 0.002015037 | 0.006286556 | 1.441257706 | 1.066177055 | ENSG00000060138.13 |
| TPM3 | 0.001623983 | 0.004052963 | 1.348659787 | 0.770537177 | ENSG00000143549.21 |
| GTF2A2 | 0.004738066 | 0.020776414 | 1.344741721 | 1.014078172 | ENSG00000140307.11 |
| ABCF1 | 0.031524009 | 0.023917788 | 1.329112304 | 1.240684479 | ENSG00000204574.13 |
| MMADHC | 0.013397565 | 0.022872616 | 1.298784264 | 1.001314567 | ENSG00000168288.13 |
| ELOC | 0.006520295 | 0.028392187 | 1.247419098 | 0.772137646 | ENSG00000154582.17 |
| MRPS15 | 0.022655334 | 0.001346038 | 1.153490068 | 1.462521587 | ENSG00000116898.12 |
| MRPL48 | 0.034782102 | 0.036301605 | 1.125468429 | 1.328277295 | ENSG00000175581.14 |
| EIF2S1 | 0.018459803 | 0.015160436 | 1.063443713 | 1.070490421 | ENSG00000134001.14 |
| FLRT2 | 0.047935374 | 8.12E−04 | −0.783497296 | −2.365857018 | ENSG00000185070.11 |
| HELLPAR | 0.010004523 | 0.003711706 | −0.95354609 | −1.708083177 | ENSG00000281344.1 |
| FNIP2 | 0.038939061 | 0.012800314 | −1.059753306 | −2.011692902 | ENSG00000052795.13 |
| CCDC26 | 0.00525569 | 0.012800314 | −1.177384541 | −1.688098154 | ENSG00000229140.11 |
| PRNCR1 | 0.026381855 | 0.028688548 | −1.182440952 | −2.205336039 | ENSG00000282961.1 |
| SYNPO2 | 0.001399149 | 0.002164273 | −1.445676213 | −2.317631854 | ENSG00000172403.11 |
| LRRC3B | 0.028157978 | 0.010897558 | −1.482046401 | −0.352666689 | ENSG00000179796.12 |
| ZBED3-AS1 | 0.015901978 | 0.042054623 | −1.531465894 | −2.854801814 | ENSG00000250802.7 |
| LINC02217 | 0.024783465 | 0.03979769 | −1.550848804 | −3.927200657 | ENSG00000248455.6 |
| MT-RNR2 | 2.36E−06 | 7.78E−11 | −2.089325441 | −2.841629703 | ENSG00000210082.2 |
| RNA5S1 | 0.005861338 | 0.031531509 | −2.141165092 | −3.276462803 | ENSG00000199352.1 |
| MTRNR2L1 | 2.50E−04 | 0.006286556 | −2.192913334 | −2.346702536 | ENSG00000256618.2 |
| MT-RNR1 | 5.40E−08 | 1.06E−53 | −2.343224038 | −3.778639833 | ENSG00000211459.2 |
| RN7SK | 0.001690037 | 0.007284285 | −2.393106003 | −2.475156685 | ENSG00000283293.1 |
| CPEB3 | 0.005897327 | 0.04251736 | −2.974280667 | −0.279153407 | ENSG00000107864.15 |

| gene | chr(hg38) | start | end | width | strand |
|---|---|---|---|---|---|
| AL135905.2 | chr6 | 63572472 | 63583587 | 11116 | + |
| UBE2C | chr20 | 45812576 | 45816957 | 4382 | + |
| TUBA1B | chr12 | 49127782 | 49131397 | 3616 | − |
| ARHGAP11A | chr15 | 32615144 | 32639941 | 24798 | + |
| SMC1A | chrX | 53374149 | 53422728 | 48580 | − |
| LGALS1 | chr22 | 37675636 | 37679802 | 4167 | + |
| ANLN | chr7 | 36389821 | 36453791 | 63971 | + |
| NOP56 | chr20 | 2652593 | 2658393 | 5801 | + |
| HMMR | chr5 | 163460203 | 163491941 | 31739 | + |
| LDHA | chr11 | 18394560 | 18408425 | 13866 | + |
| HMGB2 | chr4 | 173331376 | 173334432 | 3057 | − |
| CKS2 | chr9 | 89311195 | 89316703 | 5509 | + |
| CDKN3 | chr14 | 54396849 | 54420218 | 23370 | + |
| TUBA1C | chr12 | 49188736 | 49274600 | 85865 | + |
| RPL21 | chr13 | 27251309 | 27256691 | 5383 | + |
| S100A2 | chr1 | 153561108 | 153567890 | 6783 | − |
| KPNA2 | chr17 | 68035708 | 68046854 | 11147 | + |
| ACTB | chr7 | 5526409 | 5563902 | 37494 | − |
| GAPDH | chr12 | 6534512 | 6538374 | 3863 | + |
| ANXA2 | chr15 | 60347134 | 60402883 | 55750 | − |
| S100A10 | chr1 | 151982915 | 151993859 | 10945 | − |
| TUBB4B | chr9 | 137241287 | 137243707 | 2421 | + |
| ALDOA | chr16 | 30064164 | 30070457 | 6294 | + |
| MT2A | chr16 | 56608584 | 56609497 | 914 | + |
| RAN | chr12 | 130872037 | 130877678 | 5642 | + |
| CKS1B | chr1 | 154974653 | 154979251 | 4599 | + |
| TMSB4X | chrX | 12975110 | 12977227 | 2118 | + |
| UBE2S | chr19 | 55399745 | 55407788 | 8044 | − |
| GINS2 | chr16 | 85676198 | 85690073 | 13876 | − |
| RPL23A | chr17 | 28719985 | 28724359 | 4375 | + |
| TAGLN2 | chr1 | 159918107 | 159925507 | 7401 | − |
| RPS2P5 | chr12 | 118246084 | 118246962 | 879 | + |
| PGK1 | chrX | 77910739 | 78129295 | 218557 | + |
| CALM2 | chr2 | 47160083 | 47176921 | 16839 | − |
| FABP5 | chr8 | 81280536 | 81284777 | 4242 | + |
| ENO1 | chr1 | 8861000 | 8879190 | 18191 | − |
| ANP32E | chr1 | 150218417 | 150236156 | 17740 | − |
| TXN | chr9 | 110243810 | 110256507 | 12698 | − |
| SH3BGRL3 | chr1 | 26280086 | 26281522 | 1437 | + |
| PGAM1 | chr10 | 97426191 | 97433444 | 7254 | + |
| RPLP1 | chr15 | 69452814 | 69456205 | 3392 | + |
| RBM25 | chr14 | 73058532 | 73123899 | 65368 | + |
| GTF3A | chr13 | 27424619 | 27435823 | 11205 | + |
| ESF1 | chr20 | 13714322 | 13784886 | 70565 | − |
| SRSF7 | chr2 | 38743599 | 38751494 | 7896 | − |
| TFDP1 | chr13 | 113584721 | 113641473 | 56753 | + |
| RPS2 | chr16 | 1962058 | 1964841 | 2784 | − |
| TPX2 | chr20 | 31739271 | 31801805 | 62535 | + |
| HNRNPA2B1 | chr7 | 26173057 | 26201529 | 28473 | − |
| BOLA3 | chr2 | 74135400 | 74147912 | 12513 | − |
| UBE2N | chr12 | 93405673 | 93441947 | 36275 | − |
| SNRPE | chr1 | 203861599 | 203871152 | 9554 | + |
| RPS3 | chr11 | 75399515 | 75422280 | 22766 | + |
| STMN1 | chr1 | 25884181 | 25906991 | 22811 | − |
| KRT18 | chr12 | 52948871 | 52952906 | 4036 | + |
| UACA | chr15 | 70654554 | 70763558 | 109005 | − |
| CCNB1 | chr5 | 69167135 | 69178245 | 11111 | + |
| TMA7 | chr3 | 48440257 | 48444208 | 3952 | + |
| RPL41 | chr12 | 56116590 | 56117967 | 1378 | + |
| METAP1 | chr4 | 98995659 | 99062809 | 67151 | + |
| S100A16 | chr1 | 153606886 | 153613145 | 6260 | − |
| RPS25 | chr11 | 119015712 | 119018691 | 2980 | − |
| BNIP3 | chr10 | 131966455 | 131982013 | 15559 | − |
| RPSA | chr3 | 39406716 | 39412542 | 5827 | + |
| RPL7 | chr8 | 73290242 | 73295789 | 5548 | − |
| RPS27A | chr2 | 55231903 | 55235853 | 3951 | + |
| UBE2V1 | chr20 | 50081124 | 50115959 | 34836 | − |
| RPS20 | chr8 | 56067295 | 56074581 | 7287 | − |
| UTP18 | chr17 | 51260546 | 51297936 | 37391 | + |
| PCLAF | chr15 | 64364304 | 64387687 | 23384 | − |
| VDAC1 | chr5 | 133971871 | 134004975 | 33105 | − |
| SKA2 | chr17 | 59109857 | 59155260 | 45404 | − |
| CCND1 | chr11 | 69641156 | 69654474 | 13319 | + |
| RPS18 | chr6 | 33272075 | 33276511 | 4437 | + |
| ANXA5 | chr4 | 121667946 | 121696995 | 29050 | − |
| MRPL13 | chr8 | 120380761 | 120445402 | 64642 | − |
| RPL12 | chr9 | 127447674 | 127451406 | 3733 | − |
| RPLP0 | chr12 | 120196699 | 120201235 | 4537 | − |
| CENPF | chr1 | 214603195 | 214664571 | 61377 | + |
| RPL18A | chr19 | 17859910 | 17864153 | 4244 | + |
| FTL | chr19 | 48965309 | 48966879 | 1571 | + |
| PRELID1 | chr5 | 177303799 | 177306949 | 3151 | + |
| UQCRC1 | chr3 | 48599002 | 48610976 | 11975 | − |
| SNRPA1 | chr15 | 101281510 | 101295282 | 13773 | − |
| NME1 | chr17 | 51153559 | 51162428 | 8870 | + |
| EEF1B2 | chr2 | 206159585 | 206162928 | 3344 | + |
| RPL9 | chr4 | 39452587 | 39458931 | 6345 | − |
| RPS17 | chr15 | 82536750 | 82540459 | 3710 | − |
| PFN1 | chr17 | 4945652 | 4949061 | 3410 | − |
| AC243919.1 | chr15 | 82372196 | 82372912 | 717 | − |
| RPL32 | chr3 | 12834485 | 12841582 | 7098 | − |
| HMGB1 | chr13 | 30456704 | 30617597 | 160894 | − |
| RPS3A | chr4 | 151099624 | 151104642 | 5019 | + |
| NDUFS5 | chr1 | 39026318 | 39034636 | 8319 | + |
| BIRC5 | chr17 | 78214186 | 78225636 | 11451 | + |
| RPL35 | chr9 | 124857880 | 124861981 | 4102 | − |
| RPL5 | chr1 | 92832013 | 92841924 | 9912 | + |
| NME2 | chr17 | 51165435 | 51171744 | 6310 | + |
| KRT8 | chr12 | 52897187 | 52949954 | 52768 | − |
| ARPC1A | chr7 | 99325898 | 99366262 | 40365 | + |
| PTMA | chr2 | 231706895 | 231713541 | 6647 | + |
| RPS14 | chr5 | 150442635 | 150449739 | 7105 | − |
| SNU13 | chr22 | 41673933 | 41690504 | 16572 | − |
| C1QBP | chr17 | 5432777 | 5448830 | 16054 | − |
| HNRNPA1 | chr12 | 54280193 | 54287088 | 6896 | + |
| LYAR | chr4 | 4267701 | 4290154 | 22454 | − |
| RPS6 | chr9 | 19375715 | 19380236 | 4522 | − |
| EIF5B | chr2 | 99337371 | 99401326 | 63956 | + |
| PXMP2 | chr12 | 132687587 | 132704985 | 17399 | + |
| RPS7 | chr2 | 3575260 | 3580920 | 5661 | + |
| CALM1 | chr14 | 90396502 | 90408268 | 11767 | + |
| CNBP | chr3 | 129167827 | 129183922 | 16096 | − |
| EIF5 | chr14 | 103333544 | 103345025 | 11482 | + |
| ATP5ME | chr4 | 672436 | 674330 | 1895 | − |
| PNN | chr14 | 39175183 | 39183220 | 8038 | + |
| SNRPN | chr15 | 24823637 | 24978723 | 155087 | + |
| HSBP1 | chr16 | 83807978 | 83819737 | 11760 | + |
| GCSH | chr16 | 81081945 | 81096395 | 14451 | − |
| MAD2L1 | chr4 | 120055623 | 120066858 | 11236 | − |
| VDAC3 | chr8 | 42391624 | 42405937 | 14314 | + |
| RIDA | chr8 | 98102344 | 98117171 | 14828 | − |
| FTH1 | chr11 | 61959718 | 61967634 | 7917 | − |
| SNHG16 | chr17 | 76557764 | 76565348 | 7585 | + |
| HSPD1 | chr2 | 197486584 | 197516737 | 30154 | − |
| MAL2 | chr8 | 119165034 | 119245673 | 80640 | + |
| COTL1 | chr16 | 84565596 | 84618078 | 52483 | − |
| PA2G4 | chr12 | 56104537 | 56113910 | 9374 | + |
| HSP90AA1 | chr14 | 102080742 | 102139699 | 58958 | − |
| RPL14 | chr3 | 40457292 | 40468587 | 11296 | + |
| JPT1 | chr17 | 75135248 | 75168281 | 33034 | − |
| CCT8 | chr21 | 29055805 | 29073797 | 17993 | − |
| AC125611.3 | chr12 | 49265156 | 49273306 | 8151 | − |
| CCT5 | chr5 | 10249929 | 10266389 | 16461 | + |
| LDHB | chr12 | 21635342 | 21757857 | 122516 | − |
| UBE2D3 | chr4 | 102794383 | 102868896 | 74514 | − |
| RPS12 | chr6 | 132814569 | 132817564 | 2996 | + |
| OCIAD2 | chr4 | 48885019 | 48906937 | 21919 | − |
| SELENOF | chr1 | 86862445 | 86914424 | 51980 | − |
| MRPL39 | chr21 | 25585656 | 25607517 | 21862 | − |
| UBE2V2 | chr8 | 48008415 | 48064708 | 56294 | + |
| SPTSSA | chr14 | 34432788 | 34462240 | 29453 | − |
| DYNC1LI2 | chr16 | 66720893 | 66751609 | 30717 | − |
| OSTC | chr4 | 108650585 | 108667820 | 17236 | + |
| PSMA6 | chr14 | 35278633 | 35317493 | 38861 | + |
| NIFK | chr2 | 121726945 | 121736911 | 9967 | − |
| NUSAP1 | chr15 | 41320794 | 41381050 | 60257 | + |
| YBX3 | chr12 | 10699089 | 10723323 | 24235 | − |
| TPM3 | chr1 | 154155304 | 154194648 | 39345 | − |
| GTF2A2 | chr15 | 59638062 | 59657541 | 19480 | − |
| ABCF1 | chr6 | 30571393 | 30597179 | 25787 | + |
| MMADHC | chr2 | 149569637 | 149587778 | 18142 | − |
| ELOC | chr8 | 73939169 | 73972287 | 33119 | − |
| MRPS15 | chr1 | 36455718 | 36464384 | 8667 | − |
| MRPL48 | chr11 | 73787872 | 73865133 | 77262 | + |
| EIF2S1 | chr14 | 67360151 | 67386516 | 26366 | + |
| FLRT2 | chr14 | 85530144 | 85654428 | 124285 | + |
| HELLPAR | chr12 | 102197585 | 102402596 | 205012 | + |
| FNIP2 | chr4 | 158769026 | 158908050 | 139025 | + |
| CCDC26 | chr8 | 128634199 | 129683770 | 1049572 | − |
| PRNCR1 | chr8 | 127079874 | 127092600 | 12727 | + |
| SYNPO2 | chr4 | 118850688 | 119061247 | 210560 | + |
| LRRC3B | chr3 | 26622806 | 26717537 | 94732 | + |
| ZBED3-AS1 | chr5 | 77086688 | 77166909 | 80222 | + |
| LINC02217 | chr5 | 17403889 | 17443619 | 39731 | + |
| MT-RNR2 | chrM | 1671 | 3229 | 1559 | + |
| RNA5S1 | chr1 | 228610268 | 228610386 | 119 | − |
| MTRNR2L1 | chr17 | 22523111 | 22524663 | 1553 | + |
| MT-RNR1 | chrM | 648 | 1601 | 954 | + |
| RN7SK | chr6 | 52995621 | 52995948 | 328 | + |
| CPEB3 | chr10 | 92046692 | 92291078 | 244387 | − |

### Discussion

Cancer cell-secreted exRNAs are potential biomarker candidates with mutation- and tissue-specific signatures for cancer detection. In this work, we identified potential KRAS^{G12C}-specific exRNA biomarkers for LUAD by comprehensively analyzing the transcriptomic landscape of EVs released by LUAD-derived lung cancer cells. Our exRNA-seq analysis also showed the advantages of comparing two different methodologies: affinity column and microfluidic size-based EV isolation approaches from H358 KRAS^{G12C} mutant lung cancer cells. exRNAs reflect biological processes and mechanisms related to KRAS signaling, suggesting that they provide a snapshot of intracellular gene expression dynamics in response to alterations in mutant KRAS signaling. Interestingly, the exRNA enriched during AMG 510-mediated KRAS inhibition was significantly more variable, with strong noncoding signals derived from both lncRNAs and TE RNAs. Furthermore, the KRASG12C-dependent exRNA signatures detected across both EV isolation platforms were shown to reflect enriched RNA signatures in KRAS^{G12C} LUAD tumors from TCGA.

To map KRASG12C-dependent exRNAs, KRAS inhibition experiments were performed based on results presented by Canon et al. We detected an increase in higher molecular weight KRAS via immunoblotting, indicating AMG-bound KRAS^{G12C} (FIG. 1C) 6. Additionally, there was a marked decrease in cell viability upon treatment with AMG 510. Our AMG 510-treated cells did not exhibit a decrease in phosphorylated ERK (PERK), suggesting that either the KRAS^{WT} allele was compensating for the loss of functional KRAS^{G12C} or that the cells were acquiring an adaptive resistance to KRAS^{G12C} inhibition^{9,10,31} Notably, our exRNA analysis revealed the expected loss of RAS-dependent processes in the KRASG12C-inhibited context, supporting the notion that adaptive resistance mechanisms to KRAS^{G12C} inhibition can be detected via exRNA sequencing³²⁻³⁹.

The exRNA landscape varies based on EV subpopulations isolated by different approaches. We sought to investigate two orthogonal approaches to EV isolation, which yielded distinct EV populations with variable exRNA content. This highlights the importance of upstream EV isolation procedures before the exRNA sequencing pipeline. The ExoTIC platform is a modular, size-based EV isolation tool that enabled the capture of EVs centered around 150 nm and 120 nm with and without AMG 510 treatment, respectively¹⁹. The size distribution of EVs captured by ExoTIC overlaps significantly with the expected sizes of exosomes, 30-150 nm, a subtype of small EVs secreted by most cell types and implicated as carriers of potentially informative cancer biomarkers⁴⁰⁻⁴⁴. The significant shift in the maxima of the EV size distribution corresponding to AMG 510 treatment was accompanied by local maxima around particles of even smaller dimensions. Further EV size-fractionated exRNA sequencing will be necessary to determine the contribution of the different EV subpopulations to our exRNA data.

Alternatively, the ExoRNeasy approach utilizes proprietary, affinity-based EV isolation and enabled the capture the EVs centered around 230-270 nm, dependent on AMG 510 treatment that progressively reduced the peak size¹⁸. This size range is slightly larger than what we previously observed using ExoRNeasy to isolate EVs from a lung airway epithelial cell line, where introduction of mutant KRAS caused a modest increase in the observed EV size²⁶. These distributions are centered just beyond the canonically recognized size range of exosomes but nonetheless retain significant overlap^{33,39,40,43,44}. Both platforms captured a trend suggesting that KRAS^{G12C} inhibition corresponded with a decrease in the size of secreted EVs. This may provide more evidence for a direct role of the RAS pathway in EV and exosome biogenesis, shown previously via inhibition of Ras/Raf/ERK1/2 with Manumycin A and Rab13 regulation of 40-150 nm EV secretion^{25,45,46.}

Our exRNA sequencing pipeline captures full-length, polyadenylated RNAs encapsulated in the EVs isolated by different methods. We showed that ExoTIC captures a more complex array of exRNA molecules than ExoRNeasy and that AMG 510 treatment had no significant impact on sequencing complexity. Consistent with our previous work²⁶, there was a significant fraction of non-protein-coding RNAs sequenced from EVs, particularly lncRNAs and retained introns. This might be expected due to the different size distribution of the EVs captured, as ExoTIC libraries had relatively more noncoding transcripts detected than ExoRNeasy libraries. We observed a dramatic change to the library complexity upon the inclusion of TE insertion sequences to the alignment references. Similar to our previous work²⁶, there is a significant amount of TE-derived sequences present in the exRNA population. TEs are a diverse class of genetic elements that comprise a significant fraction of the human genome^{28,47}. Canonically silenced, they are observed to become hypomethylated in some cancers, transcribed, and contribute to pathological events in certain malignancies^{29,48-51}. Both EV isolation platforms demonstrated substantial amounts of TE exRNAs, and notably both have strong agreement in differentially enriched TE RNAs, with the vast majority of TE RNAs enriched upon AMG 510 treatment. Two TEs in particular were enriched in both EV contexts: HERV9NC-int and its associated long terminal repeat (LTR), LTR12C⁵². This suggests a KRASG12C-dependent activation of this LTR promoter, which agrees with previous observations of KRAS^{G12C} and TP53^{-/-}-mediated LTR activation⁵³⁻⁵⁵

The transcriptional events consistently observed during KRAS^{G12C} inhibition suggest a more variable response when compared to the KRASG12C-driven transcriptome. The presence of mitochondrially encoded RNAs (mtRNAs) may be a response to the loss of KRAS^{G12C} and its corresponding effects; mtRNA expression has been associated with RAS pathway expression in prostate cancer and with cell cycle regulation (MYC, E2F, and G2M signaling) in multiple cancers⁵⁶. Polyadenylation of human mtRNA is also associated with its degradation, which may indicate that this signal is one of decreased mt function⁵⁷. Additionally, lncRNAs enriched via KRAS^{G12C} inhibition do not overlap with much consistency, suggesting a stochastic nature to their upregulation upon AMG 510 treatment. Of those that are shared and characterized, PRNCR1 and CCDC26 are both in the q24 locus of chromosome 8 along with MYC.

Finally, we explored the potential utility of KRAS^{G12C}-specific exRNAs as predictive biomarkers for LUAD clinical outcomes. We evaluated our exRNA-seq findings with publicly available TCGA databases of healthy lung samples versus KRAS^{G12C} LUAD. Consensus upregulated genes across ExoRNeasy, ExoTIC, and TCGA LUAD datasets revealed a 20-gene panel capable of clustering KRAS^{G12C} LUAD samples from their WT, healthy counterparts. The genes include ENO1, GAPDH, LDHA, UBE2S, CDKN3, KPNA2, ARHGAP11A, CENPF, ANLN, TPX2, HMMR, CCNB1, MAD2L1, BIRC5, GINS2, and UBE2C. In aggregate, elevated expression of these genes was associated with significant reduction in overall survival probability in TCGA LUAD patients. This suggests that the exRNA sequencing platforms demonstrated here can be used to assay for clinically relevant diagnostic gene expression panels. Importantly, these genes appear to have interpretable connections to RAS signaling and further suggest that exRNAs are regulated in a predictable manner by oncogenic mutations to KRAS.

In conclusion, our results identified extracellular KRAS^{G12C}-specific RNA signatures that may serve as diagnostic and prognostic biomarkers for LUAD. In addition, we identified exRNA biotypes related to a clinically important KRAS-targeted inhibitor. Taken together, our findings contribute to our growing understanding of the relationship between cancer-secreted exRNAs and cancer clinical outcomes.

### Materials and Methods

Cell Lines. H358 lung cancer cell lines with KRAS G12C mutation were cultured in RPMI 1640 medium (Invitrogen) supplemented with 10% fetal bovine serum (Sigma) at 37° C., 5% CO2 in a humidified incubator. All cell lines tested negative for mycoplasma. The cell lines were purchased from American Type Culture Collection (ATCC).

Cell viability assays. For adherent viability assays, 2.5E+04 cells/well were seeded in 96-well plates and incubated at 37° C., 5% CO2 for 16 hours. Then serially-diluted AMG 510 and DMSO were added to the cells, and plates were incubated in standard culture conditions for 72 hours. Cell viability was measured using a CellTiter-Glo® Luminescent Cell Viability Assay kit (Promega) according to the manufacturer's protocol. The luminescence signal of treated samples was normalized to DMSO control. For spheroid viability assays, 5.0E+04 cells/wells were seeded in individual ultra-low adhesion 96-well plates (Corning) and incubated at 37° C., 5% CO2 for 24 hours. The cells were grown in standard culture conditions for 4 days. They were then harvested, and ATP production was measured using Cell TiterGLO Luminescent Cell Viability Assay (Promega) following the manufacturer's protocol. The luminescence was measured on a SpectraMax iD3 molecular devices.

Immunoblotting. Whole cell lysates were prepared from monolayer and spheroids of H358 cell lines. Monolayer H358 cells were washed with ice-cold PBS and lysed in RIPA lysis buffer containing protease (150 mM NaCl, 50 mM Tris pH 7.4, 1% Nonidet P-40, 0.5% SDS, 0.5% sodium deoxycholate). Spheroids were washed in PBS and extracted in a RIPA lysis buffer. Protein concentrations were quantified using the BCA Protein Assay Kit (Pierce). Equal amounts of the total protein (20 μg) with loading dye (LB) were loaded per lane in Mini-PROTEAN TGX Precast Protein Gels with 10% polyacrylamide gel (BioRad). After blocking with 5% (wt/vol) fat-free milk and 5% BSA in Tris-buffered saline with 0.075% Tween-20 (TBST), membranes were probed with 1:1000 diluted primary antibodies overnight at 4° C. (β-Actin Antibody HRP 47778 HRP from Santa Cruz Biotechnology, Phospho-p44/42 MAPK (Erk1/2) (Thr202/Tyr204) #9101, and K-Ras Antibody #53270 from Cell Signaling Technology). The membranes were washed three times with TBST buffer and incubated with 1:2000 dilution of horseradish peroxidase-conjugated (HRP) secondary antibodies (anti-mouse IgG #ab205719, and anti-rabbit #ab205718 from Abcam) for 1 h at room temperature. The blots were developed by the Amersham ECL Reagent (BioRad). Cells are treated with a dose titration of AMG 510 for 72 hours. Beta-actin control was run on the same membrane used for the RAS immunoblot. immunoblots of lysates from NCI-H358 cells treated with DMSO and 0.1 μM AMG 510 for 72 hours.

RNA Isolation & Purification. Total bulk RNA was isolated from H358 cells using Quick-RNA Mini-Prep kit (Zymogen). All RNA was quantified via NanoDrop-8000 Spectrophotometer. Exosomal RNA was isolated from cell culture media by two methods: exosomes affinity column via RNeasy Maxi Kit (Qiagen) and size-based exosome filtration via Exosome Total Isolation Chip (ExoTIC) (reference paper). In both methods, conditioned media was centrifuged at 300×g 4C for 5 min to remove possible cell debris. Extracted exosome sizes were examined by Nanoparticle Tracking Analysis system (NanoSight LM10). Extracted exosomal RNA were quantified via Qubit RNA HS assay kit (Thermo). Total bulk RNA and exosomal RNA quality were examined by Agilent 2100 Bioanalyzer (Agilent).

Quantitative reverse transcription PCR. cDNA was transcribed from 50 ng of the total RNA using iScript cDNA Synthesis Kit (Bio-Rad) according to manufacturer's protocol. cDNA was diluted 20 times and run with iTaq Universal SYBR Green Supermix (Bio-Rad). The qRT-PCR reactions were run on a QuantStudio™ 12K Flex system in triplicates according to the manufacturer's protocol. Cycle Threshold (CT) values were collected using standard analysis and target genes values were normalized to HPRT as the reference gene.

Exosomal RNA sequencing. PolyA+ long RNA was amplified and sequenced from the two exosome extraction methods from adherent monolayer and spheroid H358 cell lines. 1 ng of exosomal RNA was utilized as input for the SMART-Seq HT plus Kit (TAKARA bio) according to the manufacturer's protocol. AMPure XP PCR purification kit was used to clean up and size selection of cDNA and final libraries. cDNA and library quality were determined through the High Sensitivity DNA Kit on a Bioanalyzer 2100 (Agilent Technologies). RNAseq multiplexed libraries were sequenced as NextSeq500, 150 paired end runs, 5 million read pairs.

RNA-seq analysis. exRNA reads were first trimmed with Trimmomatic (0.39) and quantified with Salmon (1.30) with an index created using version 35 of the GENCODE reference transcriptome. The resulting transcript counts were aggregated to the gene level with Tximport and normalized with DESeqII. The TE annotation was constructed by creating a fasta file based on the Hg38 repeat track hosted on the UCSC genome browser. This fasta was combined with the previously mentioned GENCODE reference and used to create a separate, comprehensive Salmon index. TCGA LUAD counts and metadata were downloaded from the UCSC Xena browser in a DESeqII-normalized format⁶⁶.

Differential Expression and Gene Set Enrichment Analysis. DESeqII was used to estimated differential expression in all contexts with a standard model employing a formula dependent only on condition (AMG vs. DMSO, tumor vs. normal): ˜ condition. Input counts were filtered to contain genes with at least 10 total counts as determined by Salmon. DESeq output was filtered to results that had an adjusted p-value of at most 0.05. Shrunken log 2 fold change values were sorted, scaled, and used to rank the differentially expressed genes as input to Gene Set Enrichment Analysis (GSEA) performed using the R package fgsea with the ‘eps’ argument set to 0.0. Gene sets were acquired from MsigDB using the R package msigdbr. GSEA results were filtered to an adjusted p-value of at most 0.05.

Sample clustering and dimensionality reduction. Hierarchical clustering was performed with the R package pheatmap using scaled, centered DESeqII normalized counts. Principal component analysis was performed with the R package prcomp and utilized DESeqII normalized counts filtered to genes/TEs with a coefficient of variance greater than or equal to the median across the reference.

Kaplan-Meier survival analysis. Kaplan-Meier analysis was performed with the R package survival. TCGA LUAD samples were stratified into thirds based on their average expression of the gene set of interest. These strata were compared for differences in Overall Survival using the survfit function from survival with default parameters.

Statistical analysis. All statistical analyses were performed in the R programming language (4.0.2) provided in a Docker container by the Rocker Project. Wilcoxon and T tests were performed using the R function stat_compare_means which calls built-in R functions wilcox.test and t.test, respectively.

### REFERENCES

1 Barbacid, M. ras Genes. Annu. Rev. Biochem. 56, 779-827, doi: 10.1146/annurev.bi.56.070187.004023 (1987).
2 Prior, I. A., Hood, F. E. & Hartley, J. L. The Frequency of Ras Mutations in Cancer. Cancer Research 80, 2969-2974, doi: 10.1158/0008-5472.CAN-19-3682 (2020).
3 Simanshu, D. K., Nissley, D. V. & McCormick, F. RAS Proteins and Their Regulators in Human Disease. Cell 170, 17-33, doi: 10.1016/j.cell.2017.06.009 (2017).
4 Way, G. P. et al. Machine Learning Detects Pan-cancer Ras Pathway Activation in The Cancer Genome Atlas. Cell Reports 23, 172-180.e173, doi: 10.1016/j.celrep.2018.03.046 (2018).
5 Janes, M. R. et al. Targeting KRAS Mutant Cancers with a Covalent G12C-Specific Inhibitor. Cell 172, 578-589.e517, doi: 10.1016/j.cell.2018.01.006 (2018).
6 Canon, J. et al. The clinical KRAS (G12C) inhibitor AMG 510 drives anti-tumour immunity. Nature 575, 217-223, doi: 10.1038/s41586-019-1694-1 (2019).
7 Hallin, J. et al. The KRAS. Cancer Discov 10, 54-71, doi: 10.1158/2159-8290.CD-19-1167 (2020).
8Fakih, M. et al. Phase 1 study evaluating the safety, tolerability, pharmacokinetics (PK), and efficacy of AMG 510, a novel small molecule KRASG12C inhibitor, in advanced solid tumors. Journal of Clinical Oncology 37, 3003-3003, doi: 10.1200/JCO.2019.37.15_suppl.3003 (2019).
9 Xue, J. Y. et al. Rapid non-uniform adaptation to conformation-specific KRAS (G12C) inhibition. Nature 577, 421-425, doi: 10.1038/s41586-019-1884-x (2020).
10 Adachi, Y. et al. Epithelial-to-Mesenchymal Transition is a Cause of Both Intrinsic and Acquired Resistance to KRAS G12C Inhibitor in KRAS G12C-Mutant Non-Small Cell Lung Cancer. Clinical Cancer Research 26, 5962-5973, doi: 10.1158/1078-0432.CCR-20-2077 (2020).
11 Zhou, Z. et al. Extracellular RNA in a single droplet of human serum reflects physiologic and disease states. Proceedings of the National Academy of Sciences 116, 19200-19208, doi: 10.1073/pnas. 1908252116 (2019).
12 Toden, S. et al. Noninvasive characterization of Alzheimer's disease by circulating, cell-free messenger RNA next-generation sequencing. Science Advances 6, eabb 1654, doi: 10.1126/sciadv.abb1654 (2020).
13 Yan, Z. et al. Presymptomatic Increase of an Extracellular RNA in Blood Plasma Associates with the Development of Alzheimer's Disease. Current Biology 30, 1771-1782.e1773, doi: 10.1016/j.cub.2020.02.084 (2020).
14 Guler, G. D. et al. Detection of early stage pancreatic cancer using 5-hydroxymethylcytosine signatures in circulating cell free DNA. Nature Communications 11, 5270, doi: 10.1038/s41467-020-18965-w (2020).
15 Everaert, C. et al. Performance assessment of total RNA sequencing of human biofluids and extracellular vesicles. Scientific Reports 9, 17574, doi: 10.1038/s41598-019-53892-x (2019).
16Hulstaert, E. et al. Messenger RNA capture sequencing of extracellular RNA from human biofluids using a comprehensive set of spike-in controls. STAR Protocols 2, 100475, doi: 10.1016/j.xpro.2021.100475 (2021).
17 Hinger, S. A. et al. Diverse Long RNAs Are Differentially Sorted into Extracellular Vesicles Secreted by Colorectal Cancer Cells. Cell Rep 25, 715-725.e714, doi: 10.1016/j.celrep.2018.09.054 (2018).
18 Enderle, D. et al. Characterization of RNA from Exosomes and Other Extracellular Vesicles Isolated by a Novel Spin Column-Based Method. PLOS One 10, e0136133, doi: 10.1371/journal.pone.0136133 (2015).
19 Liu, F. et al. The Exosome Total Isolation Chip. ACS Nano 11, 10712-10723, doi: 10.1021/acsnano.7b04878 (2017).
20 Wang, J., Ma, P., Kim, D. H., Liu, B. F. & Demirci, U. Towards Microfluidic-Based Exosome Isolation and Detection for Tumor Therapy. Nano Today 37, doi: 10.1016/j.nantod.2020.101066 (2021).
21 Ratajczak, J. et al. Embryonic stem cell-derived microvesicles reprogram hematopoietic progenitors: evidence for horizontal transfer of mRNA and protein delivery. Leukemia 20, 847-856, doi: 10.1038/sj.leu.2404132 (2006).
22 Skog, J. et al. Glioblastoma microvesicles transport RNA and proteins that promote tumour growth and provide diagnostic biomarkers. Nat Cell Biol 10, 1470-1476, doi: 10.1038/ncb1800 (2008).
23 Valadi, H. et al. Exosome-mediated transfer of mRNAs and microRNAs is a novel mechanism of genetic exchange between cells. Nat Cell Biol 9, 654-659, doi: 10.1038/ncb1596 (2007).
24 Ibarra, A. et al. Non-invasive characterization of human bone marrow stimulation and reconstitution by cell-free messenger RNA sequencing. Nat Commun 11, 400, doi: 10.1038/s41467-019-14253-4 (2020).
25 Hinger, S. A. et al. Rab13 regulates sEV secretion in mutant KRAS colorectal cancer cells. Sci Rep 10, 15804, doi: 10.1038/s41598-020-72503-8 (2020).
26 Reggiardo, R. E. et al. Epigenomic reprogramming of repetitive noncoding RNAs and IFN-stimulated genes by mutant KRAS. (Genomics, 2020).
27 Liberzon, A. et al. Molecular signatures database (MSigDB) 3.0. Bioinformatics 27, 1739-1740, doi: 10.1093/bioinformatics/btr260 (2011).
28 de Koning, A. P., Gu, W., Castoe, T. A., Batzer, M. A. & Pollock, D. D. Repetitive elements may comprise over two-thirds of the human genome. PLOS Genet 7, e1002384, doi: 10.1371/journal.pgen.1002384 (2011).
29 Kong, Y. et al. Transposable element expression in tumors is associated with immune infiltration and increased antigenicity. Nat Commun 10, 5228, doi: 10.1038/s41467-019-13035-2 (2019).
30 Jang, H. S. et al. Transposable elements drive widespread expression of oncogenes in human cancers. Nat Genet 51, 611-617, doi: 10.1038/s41588-019-0373-3 (2019).
31 Akhave, N. S., Biter, A. B. & Hong, D. S. Mechanisms of Resistance to KRAS <sup>G12C</sup>—Targeted Therapy. Cancer Discovery, candisc; 2159-8290.CD-2120-1616v2152, doi: 10.1158/2159-8290.CD-20-1616 (2021).
32 Wu, H. et al. Circulating exosomal microRNA-96 promotes cell proliferation, migration and drug resistance by targeting LMO7. Journal of Cellular and Molecular Medicine 21, 1228-1236, doi: 10.1111/jcmm.13056 (2017).
33 Wei, F. et al. Exosomes derived from gemcitabine-resistant cells transfer malignant phenotypic traits via delivery of miRNA-222-3p. Molecular Cancer 16, 132, doi: 10.1186/s12943-017-0694-8 (2017).
34 Yuwen, D. L., Sheng, B. B., Liu, J., Wenyu, W. & Shu, Y. Q. MiR-146a-5p level in serum exosomes predicts therapeutic effect of cisplatin in non-small cell lung cancer. European Review for Medical and Pharmacological Sciences 21, 2650-2658 (2017).
35 Qin, X. et al. Cisplatin-resistant lung cancer cell-derived exosomes increase cisplatin resistance of recipient cells in exosomal miR-100-5p-dependent manner. International Journal of Nanomedicine Volume 12, 3721-3733, doi: 10.2147/IJN.S131516 (2017).
36 Jing, C. et al. Exosome-mediated gefitinib resistance in lung cancer HCC827 cells via delivery of miR-21. Oncology Letters, doi: 10.3892/ol.2018.8604 (2018).
37 Zhang, Y., Li, M. & Hu, C. Exosomal transfer of miR-214 mediates gefitinib resistance in non-small cell lung cancer. Biochemical and Biophysical Research Communications 507, 457-464, doi: 10.1016/j.bbrc.2018.11.061 (2018).
38 O'Neill, C., Gilligan, K. & Dwyer, R. Role of Extracellular Vesicles (EVs) in Cell Stress Response and Resistance to Cancer Therapy. Cancers 11, 136, doi: 10.3390/cancers11020136 (2019).
39 Kotrbová, A. et al. TEM ExosomeAnalyzer: a computer-assisted software tool for quantitative evaluation of extracellular vesicles in transmission electron microscopy images. J Extracell Vesicles 8, 1560808, doi: 10.1080/20013078.2018.1560808 (2019).
40 Booth, A. M. et al. Exosomes and HIV Gag bud from endosome-like domains of the T cell plasma membrane. J Cell Biol 172, 923-935, doi: 10.1083/jcb.200508014 (2006).
41 Raposo, G. & Stoorvogel, W. Extracellular vesicles: exosomes, microvesicles, and friends. J Cell Biol 200, 373-383, doi: 10.1083/jcb.201211138 (2013).
42 Zaborowski, M. P., Balaj, L., Breakefield, X. O. & Lai, C. P. Extracellular Vesicles: Composition, Biological Relevance, and Methods of Study. Bioscience 65, 783-797, doi: 10.1093/biosci/biv084 (2015).
43 Doyle, L. M. & Wang, M. Z. Overview of Extracellular Vesicles, Their Origin, Composition, Purpose, and Methods for Exosome Isolation and Analysis. Cells 8, doi: 10.3390/cells8070727 (2019).
44 Bebelman, M. P., Smit, M. J., Pegtel, D. M. & Baglio, S. R. Biogenesis and function of extracellular vesicles in cancer. Pharmacol Ther 188, 1-11, doi: 10.1016/j.pharmthera.2018.02.013 (2018).
45 Datta, A. et al. Manumycin A suppresses exosome biogenesis and secretion via targeted inhibition of Ras/Raf/ERK1/2 signaling and hnRNP H1 in castration-resistant prostate cancer cells. Cancer Lett 408, 73-81, doi: 10.1016/j.canlet.2017.08.020 (2017).
46 Sexton, R. E., Mpilla, G., Kim, S., Philip, P. A. & Azmi, A. S. Ras and exosome signaling. Semin Cancer Biol 54, 131-137, doi: 10.1016/j.semcancer.2019.02.004 (2019).
47 Bourque, G. et al. Ten things you should know about transposable elements. Genome Biol 19, 199, doi: 10.1186/s13059-018-1577-z (2018).
48 Payer, L. M. & Burns, K. H. Transposable elements in human genetic disease. Nature Reviews Genetics 20, 760-772, doi: 10.1038/s41576-019-0165-8 (2019).
49 Choi, I.-S. et al. Hypomethylation of LINE-1 and Alu in well-differentiated neuroendocrine tumors (pancreatic endocrine tumors and carcinoid tumors). Mod Pathol 20, 802-810, doi: 10.1038/modpathol.3800825 (2007).
50 Espinet, E. et al. Aggressive PDACs Show Hypomethylation of Repetitive Elements and the Execution of an Intrinsic IFN Program Linked to a Ductal Cell of Origin. Cancer Discovery 11, 638-659, doi: 10.1158/2159-8290.CD-20-1202 (2021).
51 Cañadas, I. et al. Tumor innate immunity primed by specific interferon-stimulated endogenous retroviruses. Nature Medicine 24, 1143-1150, doi: 10.1038/s41591-018-0116-5 (2018).
52 Vargiu, L. et al. Classification and characterization of human endogenous retroviruses; mosaic forms are common. Retrovirology 13, 7, doi: 10.1186/s12977-015-0232-y (2016).
53 Leonova, K. I. et al. p53 cooperates with DNA methylation and a suicidal interferon response to maintain epigenetic silencing of repeats and noncoding RNAs. Proc Natl Acad Sci USA 110, E89-98, doi: 10.1073/pnas. 1216922110 (2013).
54 Roulois, D. et al. DNA-Demethylating Agents Target Colorectal Cancer Cells by Inducing Viral Mimicry by Endogenous Transcripts. Cell 162, 961-973, doi: 10.1016/j.cell.2015.07.056 (2015).
55 Chiappinelli, K. B. et al. Inhibiting DNA Methylation Causes an Interferon Response in Cancer via dsRNA Including Endogenous Retroviruses. Cell 162, 974-986, doi: 10.1016/j.cell.2015.07.011 (2015).
56 Yuan, Y. et al. Comprehensive molecular characterization of mitochondrial genomes in human cancers. Nat Genet 52, 342-352, doi: 10.1038/s41588-019-0557-x (2020).
57 Slomovic, S., Laufer, D., Geiger, D. & Schuster, G. Polyadenylation and degradation of human mitochondrial RNA: the prokaryotic past leaves its mark. Mol Cell Biol 25, 6427-6435, doi: 10.1128/MCB.25.15.6427-6435.2005 (2005).
58 Cairns, R. A., Harris, I. S. & Mak, T. W. Regulation of cancer cell metabolism. Nat Rev Cancer 11, 85-95, doi: 10.1038/nrc2981 (2011).
59 Semenza, G. L. Hypoxia-inducible factors: mediators of cancer progression and targets for cancer therapy. Trends Pharmacol Sci 33, 207-214, doi: 10.1016/j.tips.2012.01.005 (2012).
60 Song, Y. et al. Alpha-enolase as a potential cancer prognostic marker promotes cell growth, migration, and invasion in glioma. Mol Cancer 13, 65, doi: 10.1186/1476-4598-13-65 (2014).
61 Engeland, K. Cell cycle arrest through indirect transcriptional repression by p53: I have a DREAM. Cell Death Differ 25, 114-132, doi: 10.1038/cdd.2017.172 (2018).
62 Kagawa, Y. et al. Cell cycle-dependent Rho GTPase activity dynamically regulates cancer cell motility and invasion in vivo. PLOS One 8, e83629, doi: 10.1371/journal.pone.0083629 (2013).
63 Xu, J. et al. RhoGAPs attenuate cell proliferation by direct interaction with p53 tetramerization domain. Cell Rep 3, 1526-1538, doi: 10.1016/j.celrep.2013.04.017 (2013).
64 Lin, S. C. et al. Dysregulation of miRNAs-COUP-TFII-FOXMI-CENPF axis contributes to the metastasis of prostate cancer. Nat Commun 7, 11418, doi: 10.1038/ncomms11418 (2016).
65 Bretones, G., Delgado, M. D. & León, J. Myc and cell cycle control. Biochim Biophys Acta 1849, 506-516, doi: 10.1016/j.bbagrm.2014.03.013 (2015).
66 Goldman, M. J. et al. Visualizing and interpreting cancer genomics data via the Xena platform. Nat Biotechnol 38, 675-678, doi: 10.1038/s41587-020-0546-8 (2020).

1. A method for detecting a RAS pathway mutation in a subject, the method comprising:
(a) obtaining a biological sample from the subject;
(b) isolating nucleic acids from the biological sample; and
(c) analyzing the expression level of extracellular RNAs in the nucleic acids,
wherein a differential expression level of the extracellular RNAs compared to a control sample indicates that the subject has a RAS pathway mutation.

2. The method of claim 1 where the RAS pathway mutation is in KRAS.

3. The method of claim 2, wherein the KRAS mutation is KRAS (G12C).

4. The method of claim 1, wherein the biological sample comprises extracellular vesicles isolated from biofluids from the subject.

5. The method of claim 4, wherein the biofluids comprise blood or serum.

6. The method of claim 1, wherein the extracellular vesicles comprise exosomes and/or microvesicles.

7. The method of claim 1, wherein the extracellular vesicles are greater than 200 nm in size.

8. The method of claim 1, wherein the extracellular vesicles are less than 200 nm in size.

9. The method of claim 1, wherein the nucleic acids comprise polyadenylated RNAs.

10. The method of claim 1, wherein analyzing the expression level of the extracellular RNAs comprises performing polymerase chain reaction (PCR), reverse transcriptase polymerase chain reaction (RT-PCR), single-cell RNA-sequencing, microarray analysis, a Northern blot, serial analysis of gene expression (SAGE), immunoassay, hybridization capture, cDNA sequencing, direct RNA sequencing, nanopore sequencing, mass spectrometry, a CRISPR based technology, or combinations thereof.

11. The method of claim 1, wherein analyzing the expression level of the extracellular RNAs comprises performing sequencing.

12. The method of claim 11, wherein the sequencing comprises obtaining one or more sequencing reads of the extracellular RNAs.

13. The method of claim 12, wherein the analyzing comprises aligning the sequencing reads of the extracellular RNAs to repetitive sequences in a human genome.

14. The method of claim 1, wherein isolating the nucleic acids comprises isolating extracellular vesicles from the biological sample followed by isolating the nucleic acids from the extracellular vesicles.

15. The method of claim 14, wherein the nucleic acids are extracellular RNAs from the extracellular vesicles.

16. The method of claim 13, further comprising repeating steps (a) to (c).

17. The method of claim 1, wherein analyzing the expression of the extracellular RNAs comprises analyzing the expression of BNIP3, NUSAP1, OCIAD2, KRT18, ENO1, GAPDH, LDHA, UBE2S, CDKN3, KPNA2, ARHGAP11A, CENPF, ANLN, TPX2, HMMR, CCNB1, MAD2L1, BIRC5, GINS2, and UBE2C.

18. The method of claim 1, wherein the subject has or is suspected of having cancer.

19. The method of claim 18, wherein the cancer is a RAS mutant cancer.

20. The method of claim 19, wherein the RAS mutant cancer is a lung cancer.

21. The method of claim 1, further comprising administering to the subject one or more anticancer agents.

22. The method of claim 21, wherein the anticancer agent is an inhibitor of KRAS.

23. The method of claim 22, wherein the inhibitor of KRAS is selected from the group consisting of a small molecule, a nucleic acid or an antibody.

24. The method of claim 23, wherein the small molecule is selected from the group consisting of MRTX-849, ARS1620, and AMG 510.
